(19) United States
(12) Patent Application Publication
(10) Pub. No.: US 2003/0121073 A1
(43) Pub. Date: Jun. 26, 2003

(54) Use of acetoacetyl-CoA thiolase for identifying new fungicidally active substances

(76) Inventors: Peter Schreier, Koln (DE); Ronald Ebbert, Monheim (DE); Edith Oehmen, Leichlingen (DE)

Correspondence Address: BAYER CROPSCIENCE LP, 100 BAYER ROAD, PITTSBURGH, PA, 15205, US

(21) Appl. No.: 10/305,442

(22) Filed: Nov. 26, 2002

(30) Foreign Application Priority Data
Dec. 3, 2001 (DE) 10159264.7

(51) Int. Cl. A01H 5/00; C07H 21/04; C12Q 1/18; C12N 9/10; C12N 1/16; C12N 1/18; C12N 15/82; C12N 15/74; C12N 5/04

(52) U.S. Cl. 800/279; 435/69.1; 435/254.2; 435/193; 435/320.1; 435/419; 435/483; 536/23.2; 435/32

(57) ABSTRACT

The invention relates to nucleic acids which encode fungal polypeptides with the biological activity of acetoacetyl-CoA thiolase, to the polypeptides encoded by them and to their use as targets for fungicides and to their use for identifying new, fungicidally active compounds, and to methods of finding modulators of these polypeptides and, finally, to transgenic organisms containing sequences encoding fungal polypeptides with the function of an acetoacetyl-CoA thiolase.

The invention relates to nucleic acids which encode fungal polypeptides with the biological activity of acetoacetyl-CoA thiolase, to the polypeptides encoded by them and to their use as targets for fungicides and to their use for identifying new, fungicidally active compounds, and to methods of finding modulators of these polypeptides and, finally, to transgenic organisms containing sequences encoding fungal polypeptides with the function of an acetoacetyl-CoA thiolase.

Undesired fungal growth which leads every year to considerable damage, for example in agriculture, can be controlled by the use of fungicides. The demands made on fungicides have increased constantly with regard to their activity, their costs and above all their ecological soundness. There exists therefore a demand for novel substances or classes of substances which can be developed into potent and ecologically sound novel fungicides. In general, it is customary to search for such novel lead structures in greenhouse tests. However, such tests require a high input of labour and a high financial input. The number of the substances which can be tested in the greenhouse is, accordingly, limited. An alternative to such tests is the use of what are known as (ultra) high-throughput screening methods ((U)HTS). This involves testing a large number of individual substances with regard to their effect on individual cells, individual gene products or genes in an automated method. When certain substances are found to have an effect, they can be studied in conventional screening methods and, if appropriate, developed further.

Advantageous targets for fungicides are frequently searched for in essential biosynthesis pathways. Ideal fungicides are, moreover, those substances which inhibit gene products which have a decisive importance in the manifestation of the pathogenicity for a fungus. An example of such a fungicide is, for example, the active substance carpropamid, which inhibits fungal melanin biosynthesis and thus prevents the formation of intact appressoria (adhesion organs) (Kurahashi et al., 1997). However, there is only a very small number of known gene products which play such a role for fungi. Moreover, fungicides are known which lead to auxotrophism of the target cells by inhibiting corresponding biosynthesis pathways and, as a consequence, to the loss of pathogenicity. Thus, for example, the inhibition of adenosine deaminase upon addition of ethirimol leads to a significant inhibition in Blumeria graminis.

The mevalonate pathway (cf. FIG. 1 and FIG. 2) is an essential metabolic pathway since its products include all isoprene-containing compounds such as streols, quinones, dolichol and isopentylated adenosine in tRNAs. Moreover, some proteins undergo post-translational modification by the isoprenoid groups farnesyl and geranyl. Mavalonate, the precursor of all isoprenoids, is formed in three stages (see FIG. 1).

Two classes of thiolase which can be differentiated on the basis of the chain length of their substrates for thiolytic reaction exist in eukaryotes and prokaryotes. Acetoacetyl-CoA thiolases (EC 2.3.1.9), also known as acetyl-CoA C-acetyltransferase, hereinbelow termed ACAT, are specific for acetoacetyl-CoA. In contrast, 3-ketoacyl-CoA thiolases (EC 2.3.1.16) exhibit a broad chain length spectrum for a variety of β-ketoacyl-CoA substrates. These two classes can be divided further with regard to their subcellular localization. Acetoacetyl-CoA thiolases are generally located in the cytoplasm, where they catalyse the first reaction step from acetyl-CoA to acetoacetyl-CoA in the mevalonate metabolic pathway (this being reverse reaction of the reversible thiolytic cleavage of acetoacetyl-CoA) and in mitochondria, where they exert a function in the ketone body metabolism. In alkane-assimilating fungi, peroxisomal ACATs have also been identified (Kurihara et al., 1988, Kurihara et al., 1992, Yamagami et al., 2001). In contrast, 3-ketoacyl-CoA thiolases are generally localized in mitochondria and peroxisomes and involved in fatty acid β-oxidation.

Two different acetoacetyl-CoA thiolase isoenzymes, a cytoplasmic and a mitochondrial one, have been described for Saccharomyces cerevisiae (Kornblatt and Rudney, 1971b). The cytoplasmic acetoacetyl-CoA thiolase probably acts anabolically in the mevalonate metabolism, whereas the mitochondrial acetoacetyl-CoA thiolase regulates the pool size of acetoacetyl-CoA, which is formed in the fatty acid catabolism, and of acetyl-CoA, which enters the citric acid cycle. The size of the two enzymes, as estimated by means of gel filtration, is 140,000 Da for the cytoplasmic isoenzyme and 65,000 Da for the mitochondrial isoenzyme. The two isoenzymes differ further with regard to their ability of utilizing dithiothreitol in vitro as thiol donor (Kornblatt and Rudney 1971a). No immunological data or protein sequence information exist on the degree of relatedness of the two isoenzymes.

Acetoacetyl-CoA thiolase genes have been isolated from a large number of prokaryotic and eukaryotic organisms. In bacteria, acetoacetyl-CoA thiolase exerts a function in the biosynthesis of poly-β-hydroxybutyric acid, a storage molecule. Bacterial genes which encode acetoacetyl-CoA thiolases have been isolated for example from Zoogloea ramigera (Peoples et al., 1987) and Thiocystis violacea (Liebergesell and Steinbüchel, 1993). Genes which encode mitochondrial acetoacetyl-CoA thiolases have been identified, inter alia, in human (Fukao et al., 1990) and rat cells (Fukao et al., 1989). The yeast Candida tropicalis has two ACAT isoenzymes (cytosolic and peroxisomal) which are encoded by the same two genes (CT-T1A and CT-T1B; Kanayama et al., 1997). The product of the acetoacetyl-CoA thiolase gene from the yeast Saccharomyces uvarum (Dequin et al., 1988) is probably localized in the cytoplasm.

Ergosterol is the main product of the mevalonate metabolic pathway of the yeast Saccharomyces cerevisiae (cf. FIG. 1 and FIG. 2). Ergosterol is a constituent of fungal cell membranes and a prerequisite of fungal growth. The gene ERG10 which encodes ACAT, has been described as essential gene for S. cerevisiae (cf. Hiser et al., 1994). ERG10 encodes a polypeptide with 398 amino acids and a calculated molecular mass of 41681 Da. It has been demonstrated by means of gel filtration that recombinant yeast ACAT exists as homotetramer with a protein size of approx. 179 kDa.

ACAT is thoroughly characterized biochemically (cf. Clinkenbeard et al., 1975, Dequin et al., 1988, Hiser et al., 1994). The crystal structure of the enzyme has been identified from the bacterium Zoogloea ramigera (Modis et al., 1999).

Despite this outstanding characterization of ACAT, the enzyme has never been recognized as target for fungicidal active compounds. So far, the utilization of this interesting enzyme is restricted to clinical chemistry, where ACAT is used for the diagnosis of defects in the ketone body metabolism (Watanabe et al., 1998). However, it is known that ACAT from a variety of organisms (for example in rat liver, cf. Greenspan et al., 1989) can be inhibited by organic molecules.

The complete cDNA sequence and the corresponding gene (acc1) (cf. SEQ ID NO. 1 and SEQ ID NO. 2) from Ustilago maydis encoding ACAT have been isolated within the scope of the present invention [for example encoding sequence of the Ustilago maydis strain 521, DSM No. 14603, chromosome IX BAC Um31, start 15266-15210 (exon 1), 15073-14703 (exon 2) and 14150-13356 (exon 3), 15209-15074 (intron 1) and 14702-14151 (intron 2), corresponding to a length of 408 amino acids].

The smut fungus Ustilago maydis, a Basidiomycete, attacks maize plants. The disease occurs in all areas where maize is grown, but gains importance only during dry years. Typical symptoms are the gall-like, fist-sized swellings (blisters) which are formed on all aerial plant parts. The galls are first covered by a whitish-grey coarse membrane. When the membrane ruptures, a black mass of ustilospores, which is first greasy and later powdery, is released. Further phytopathogenic species of the genus Ustilago are, for example, U. nuda (causes loose smut of barley and wheat), U. nigra (causes black smut of barley), U. hordei (causes covered smut of barley) and U. avenae (causes loose smut of oats).

By means of knock-out analyses in the Basidiomycete Ustilago maydis it has, surprisingly, been found within the scope of the present invention that the enzyme ACAT is essential for survival of the organism also in this phytopathogenic fungus, as is the case in the Ascomycete Saccharomyces cerevisiae (see also Hiser et al., 1994). This allows the conclusion that ACAT plays an important role, not only for a specific fungus, in this case Saccharomyces cerevisiae, but for fungi in general. ACAT was thus recognized for the first time as an optimal target for the search for novel, specific fungicides, precisely in phytopathogenic fungi, and it was thus made possible to identify, with the aid of this target, lead structures which may be entirely new and which inhibit ACAT and which can be used as fungicides.

It has further been found within the scope of the present invention that ACAT can be used for identifying substances in suitable test methods which affect the activity of the enzyme. In addition to an ACAT from a phytopathogenic fungus, which is characterized by its amino acid sequence and the nucleic acid sequence encoding it, suitable test methods for identifying modulators of the enzyme are also provided.

It has furthermore been found within the scope of the present invention that ACAT is indeed inhibited by active compounds and that a fungal organism treated with these active compounds can be damaged or killed by the treatment with these active compounds, that is to say that ACAT inhibitors can also be used as fungicides. For example, it is shown in the present invention that the inhibition of ACAT with substances identified in a test system leads to destruction of the treated fungi both in synthetic media and on the plant.

Even though it was already known that the gene encoding the Saccharomyces cerevisiae ACAT is an essential gene, it was previously unknown that ACAT, in fungi, can be a target protein of fungicidally active substances. Thus, it is demonstrated for the first time in the present invention that ACAT constitutes an enzyme which is vital for fungi, in particular phytopathogenic fungi, and which is therefore particularly suitable for use as target protein for the search for further, improved, fungicidally active compounds.

The proof that ACAT constitutes a vital enzyme for fungi including phytopathogenic fungi is preferably provided by the generation of deletion mutants. The method described in DE 101 33 928.3 is used for this purpose (see also the Examples).

Furthermore, the Ustilago maydis ACAT is described for the first time in the present invention. The ACAT described belongs to the above-described class of thiolases. The sequence, encoding ACAT, from a Basidiomycete was hitherto unknown. The genes for ACATs from a variety of fungi (all of them Ascomycetes), viz. Saccharomyces cervisiae, Saccharomyces bayanus, Candida tropicalis, Yarrowia lipolytica, Schizosaccharomyces pombe, all of which are not phytopathogenic, and Mycosphaerella graminicola, which is phytophathogenic, are deposited in databases. The databases furthermore contain fragments of the ACAT-encoding DNA from the phytopathogenic Ascomycete Magnaporthe grisea, the non-pathogenic Ascomycetes Neurospora crassa, Saccharomyces kluyveri and Zygosaccharomyces rouxii and the phytopathogenic Oomycete Phytophthora infestans (see Table 1).

| | | | | |
|---|---|---|---|---|
| | | Identity | Similarity | Length |
| Organism | Accession No. | (%) | (%) | (Amino acids) |

Protein sequences

| | | | | |
|---|---|---|---|---|
| Candida | SWISS-PROT | 55.4 | 64.1 | 402 |
| tropicalis | Q04677 | | | |
| Candida | SPTREMBL | 55.4 | 64.2 | 402 |
| tropicalis | Q12598 | | | |
| Saccharomyces | SWISS-PROT | 52.9 | 62 | 398 |
| bayanus | P10551 | | | |
| Saccharomyces | SWISS-PROT | 54 | 61.5 | 398 |
| cerevisiae | P41338 | | | |
| Mycosphaerella | SPTREMBL | 54.8 | 64.6 | 444 |
| graminicola | Q9Y838 | | | |
| Schizosaccharo | SPTREMBL | 54.5 | 63.8 | 395 |
| myces pombe | Q9UQW6 | | | |
| Yarrowia | SPTREMBL | 60.5 | 68.4 | 397 |
| lipolytica | Q9C1T3 | | | |

DNA Sequences

| | | | | |
|---|---|---|---|---|
| Magnaporthe | EMBL | 58.7 | 66.8 | 723 |
| grisea | AQ361828 | | | |
| Saccharomyces | EMBL | 36.4 | 42 | 987 |
| kluyveri | AL405378 | | | |
| Zygosaccharom | EMBL | 48.3 | 56.9 | 943 |
| yces rouxii | AL396273 | | | |
| Neurospora | EMBL | 59.3 | 68.3 | 444 |
| crassa | AW713418 | | | |
| Phytophthora | EMBL | 48.9 | 56.8 | 598 |
| infestans | BE777121 | | | |

Table 1: List of the fungi whose protein or DNA sequences encoding ACAT are known or from which part-sequences which were postulated as encoding parts of ACAT. Shown are the origin of the sequence information and the similarity and identity of the sequences or sequence fragments with the Ustilago maydis ACAT (amino acid level).

The putative part-sequences, known as ESTs, can now be confirmed as sequences encoding ACAT by means of the known Saccharomyces sequence and the Ustilago maydis sequence according to the invention.

Based on the comparisons carried out within the scope of the present invention (see also Table 1), it can now be stated that the Ustilago maydis ACATs and the ACATs from other fungi such as, for example, Saccharomyces cerevisiae or Yarrowia lipolytica have a considerable degree of homology with each other, which is why polypeptides which are homologous to the Ustilago maydis ACAT and which are encoded by correspondingly homologous nucleic acids can also be used as partners for molecular interactions (targets) of fungicidal active compounds. The homologous nucleic acids or polypeptides from phytopathogenic fungi are of particular interest. Phytopathogenic Basidiomycetes can be used especially preferably for this purpose. Owing to the high degree of homology between the ACATs, the ACATs from fungi which are pathogenic to humans (see Table 1) or the nucleic acids encoding them may also be used for identifying inhibitors of the enzyme. Analogously, ACAT inhibitors may also display an activity against fungi which are pathogenic to humans and may be used as antimycotics.

This is why the present invention fully encompasses the use of fungal ACATs, in particular from Ascomycetes, Basidiomycetes and Oomycetes, very particularly from phytopathogenic fungi or phytopathogenic Basidiomycetes, and in particular from Ustilago maydis for identifying fungicidally active substances.

The abovementioned homologous polypeptides especially preferably take the form of those which have at least 60%, preferably 80%, especially preferably 90%, very especially preferably 95% similarity with the Ustilago maydis ACAT over a length of at least 20, preferably at least 25, especially preferably at least 30 and very especially preferably at least 100 consecutive amino acids and most preferably over the entire length.

Such polypeptides which are homologous to the Ustilago maydis ACAT, in particular to the polypeptide of SEQ ID NO. 4 and which can be used for identifying fungal active substances need not constitute complete fungal ACATs, but may also only constitute fragments of these as long as they at least still have a biological activity of the complete fungal ACATs. Polypeptides which exert the same type of biological activity as an ACAT with an amino acid sequence as shown in SEQ ID NO. 4 are still considered as being according to the invention. In this context, the polypeptides according to the invention need not be deducible from ACATs from Ustilago maydis or from phytopathogenic fungi, for the abovementioned reasons. Polypeptides which are considered according to the invention are, above all, also those polypeptides which correspond to ACATs for example of the following fungi, or fragments of these, and which still have their biological activity:

Plasmodiophoromycetes, Oomycetes, Chytridiomycetes, Zygomycetes, Ascomycetes, Basidiomycetes and Deuteromycetes, for example.

Pythium species such as, for example, Pythium ultimum, Phytophthora species such as, for example, Phytophthora infestans, Pseudoperonospora species such as, for example, Pseudoperonospora humuli or Pseudoperonospora cubensis, Plasmopara species such as, for example, Plasmopara viticola, Bremia species such as, for example, Bremia lactucae, Peronospora species such as, for example, Peronospora pisi or P. brassicae, Erysiphe species such as, for example, Erysiphe graminis, Sphaerotheca species such as, for example, Sphaerotheca fuliginea, Podosphaera species such as, for example, Podosphaera leucotricha, Venturia species such as, for example, Venturia inaequalis, Pyrenophora species such as, for example, Pyrenophora teres or P. graminea (conidial form: Drechslera, syn: Helminthosporium), Cochliobolus species such as, for example, Cochliobolus sativus (conidial form: Drechslera, syn: Helminthosporium), Uromyces species such as, for example, Uromyces appendiculatus, Puccinia species such as, for example, Puccinia recondita, Sclerotinia species such as, for example, Sclerotinia sclerotiorum, Tilletia species such as, for example, Tilletia caries; Ustilago species such as, for example, Ustilago nuda or Ustilago avenae, Pellicularia species such as, for example, Pellicularia sasakii, Pyricularia species such as, for example, Pyricularia oryzae, Fusarium species such as, for example, Fusarium culmorum, Botrytis species, Septoria species such as, for example, Septoria nodorum, Leptosphaeria species such as, for example, Leptosphaeria nodorum, Cercospora species such as, for example, Cercospora canescens, Alternaria species such as, for example, Alternaria brassicae or Pseudocercosporella species such as, for example, Pseudocercosporella herpotrichoides.

Others which are of particular interest are, for example, Magnaporthe grisea, Cochliobulus heterostrophus, Nectria haematococca and Phytophthora species.

Fungicidal active compounds which are found with the aid of the ACATs according to the invention may also interact with ACATs from fungal species which are pathogenic to humans; however, the interaction with the different ACATs which appear in these fungi need not always be equally pronounced.

The present invention therefore also relates to the use of ACAT inhibitors for the preparation of compositions for treating diseases caused by fungi which are pathogenic to humans.

Of particular interest in this context are the following fungi which are pathogenic to humans and which may cause the symptoms stated hereinbelow:

Dermatophytes such as, for example, Trichophyton spec., Microsporum spec., Epidermophyton floccosum or Keratomyces ajelloi, which cause, for example, athlete's foot (tinea pedis),

Yeasts such as, for example, Candida albicans, which causes soor oesophagitis and dermatitis, Candida glabrata, Candida krusei or Cryptococcus neoformans, which may cause, for example, pulmonal cryptococcosis or else torulosis,

Moulds such as, for example, Aspergillus fumigatus, A. flavus, A. niger, which cause, for example, bronchopulmonary Aspergillosis or fungal sepsis, Mucor spec., Absidia spec., or Rhizopus spec., which cause, for example, zygomycoses (intravasal mycoses), Rhinosporidium seeberi, which causes, for example, chronic granulomatous pharyngitis and tracheitis, Madurella myzetomatis, which causes, for example, subcutaneous mycetomes, Histoplasma capsulatum, which causes, for example, reticuloendothelial cytomycosis and Darling's disease, Coccidioides immitis, which causes, for example, pulmonary coccidioidomycosis and sepsis, Paracoccidioides brasiliensis, which causes, for example, South American blastomycosis, Blastomyces dermatitidis, which causes, for example, Gilchrist's disease and North American blastomycosis, Loboa loboi, which causes, for example, keloid blastomycosis and Lobo's disease, and Sporothrix schenckii, which causes, for example, sporotrichosis (granulomatous dermal mycosis).

Fungicidal active compounds which are found with the aid of the ACATs according to the invention can therefore also interact with ACATs from a large number of other phytopathogenic fungal species; the interaction with the different ACATs which occur in these fungi need not always be equally pronounced. This explains, inter alia, the selectivity which has been observed of the substances which are active on this enzyme.

The present invention therefore relates to nucleic acids which encode complete ACATs from phytopathogenic fungi, with the exception of the sequence fragments from Blumeria graminis, Cladosporium fulvum and Mycosphaerella graminicola which are listed in Table 1 and which have the sequences deposited under the stated accession numbers.

The present invention particularly relates to nucleic acids which encode ACATs from Basidiomycetes, preferably from phytopathogenic Basidiomycetes, very especially preferably from the genus Ustilago.

The present invention very especially preferably relates to nucleic acids which encode Ustilago maydis ACAT.

The present invention especially preferably relates to Ustilago maydis nucleic acids as shown in SEQ ID NO. 2 and SEQ ID NO. 3, which encode a polypeptide as shown in SEQ ID NO. 4 or active fragments thereof.

In particular, the nucleic acids according to the invention take the form of single-stranded or double-stranded deoxyribonucleic acids (DNA) or ribonucleic acids (RNA). Preferred embodiments are fragments of genomic DNA, which may contain introns, and cDNAs.

The nucleic acids according to the invention preferably take the form of DNA fragments which correspond to the cDNA of the nucleic acids according to the invention.

The nucleic acids according to the invention especially preferably comprise a fungal sequence selected from

a) the sequence as shown in SEQ ID NO. 1, SEQ ID NO. 2 and SEQ ID NO.3,

b) sequences encoding a polypeptide comprising the amino acid sequence as shown in SEQ ID NO. 4,

c) sequences which hybridize with the sequences defined under a) and/or b) at a hybridization temperature of 35-52° C.,

d) sequences with at least 60%, preferably 80%, especially preferably 90% and very especially preferably 95% identity with the sequences defined under a) and/or b),

e) sequences which are complementary to the sequences defined under a) and/or b), and

f) sequences which, owing to the degeneracy of the genetic code, encode the same amino acid sequence as the sequences defined under a) to e).

A cDNA molecule with the sequence as shown in SEQ ID NO. 1, SEQ ID NO. 2 or SEQ ID NO. 3 encoding the Ustilago maydis ACAT with the SEQ ID NO. 4 constitutes a very especially preferred embodiment of the nucleic acids according to the invention.

The term “complete” ACAT as used in the present context describes the ACATs encoded by the complete coding region of a transcription unit, starting with the ATG start codon and comprising all the information-bearing exon regions of the gene encoding ACAT which is present in the source organism, as well as the signals required for correct transcriptional termination.

The term “active fragment” as used in the present context describes nucleic acids encoding ACAT which are no longer complete, but still encode enzymes with the biological activity of an ACAT and which are capable of catalysing a reaction characteristic of ACAT, as described above. Such fragments are shorter than the above-described complete nucleic acids encoding ACAT. In this context, nucleic acids may have been removed both up to the 3′ and/or 5′ ends of the sequence, or else parts of the sequence which do not have a decisive adverse effect on the biological activity of ACAT may have been deleted, i.e. removed. A lower or else, if appropriate, an increased activity, which still allows the characterization or use of the resulting ACAT fragment, is considered as sufficient for the purposes of the term as used herein. The term “active fragment” may likewise refer to the amino acid sequence of ACAT; in this case, it applies analogously to what has been said above to those polypeptides which no longer contain certain portions in comparison with the above-described complete sequence, but where no decisive adverse effect is exerted on the biological activity of the enzyme.

The term “gene” as used in the present context is the name for a segment from the genome of a cell which is responsible for the synthesis of a polypeptide chain.

The term “to hybridize” as used in the present context describes the process in which a single-stranded nucleic acid molecule undergoes base pairing with a complementary strand. For example, starting from the sequence information which is mentioned herein or which can be deduced, DNA fragments can be isolated, in this manner, from phytopathogenic fungi other than Ustilago maydis, which fragments encode ACATs with the same properties as or similar properties to one of the ACATs according to the invention.

Hybridization conditions are calculated approximately by the following formula:

The melting temperature Tm=81.5° C.+16.6 log[c(Na⁺)]+0.41(% G+C)−500/n

(Lottspeich and Zorbas, 1998).

In this formula, c is the concentration and n the length of the hybridizing sequence segment in base pairs. For a sequence>100 bp, the term 500/n is dropped. The highest stringency involves washing at a temperature of 5-15° C. below Tm and an ionic strength of 15 mM Na⁺ (corresponds to 0.1×SSC). If an RNA sample is used for hybridization, the melting point is 10-15° C. higher.

Preferred hybridization conditions are stated hereinbelow:

Hybridization solution: DIG Easy Hyb (Roche, ZZ) hybridization temperature: 37° C. to 50° C., preferably 42° C. (DNA-DNA), 50° C. (DNA-RNA).

Wash step 1: 2×SSC, 0.1% SDS 2×5 min at room temperature;

Wash step 2: 1×SSC, 0.1% SDS 2×15 min at 50° C.; preferably 0.5×SSC, 0.1% SDS 2×15 min at 65° C.; especially preferably 0.2×SSC, 2×15 min at 68° C.

The degree of identity of the nucleic acids /peptides is preferably determined with the aid of the program NCBI BLASTN Version 2.0.4. (Altschul et al., 1997).

The term “heterologous promoter” as used in the present context refers to a promoter with properties other than the promoter which controls the expression of the gene in question in the original organism.

The choice of heterologous promoters depends on whether prokaryotic or eukaryotic cells or cell-free systems are used for expression. Examples of heterologous promoters are the cauliflower mosaic virus 35S promoter for plant cells, the alcohol dehydrogenase promoter for yeast cells, the T3, T7 or SP6 promoters for prokaryotic cells or cell-free systems, and tissue-specific promoters from phytopathogenic fungi, for example the specific promoter of the aldolase to be used in accordance with the invention.

The present invention furthermore relates to vectors containing a nucleic acid according to the invention, a regulatory region according to the invention or a DNA construct according to the invention.

Vectors which can be used are all those phages, plasmids, phagemids, phasmids, cosmids, YACs, BACs, artificial chromosomes or particles suitable for particle bombardment which are used in molecular-biological laboratories.

A preferred vector is pET15b (Novagen).

The present invention also relates to host cells containing a nucleic acid according to the invention, a DNA construct according to the invention or a vector according to the invention.

The term “host cell” as used in the present context refers to cells which do not naturally contain the nucleic acids according to the invention.

Suitable as host cells are prokaryotic cells, preferably E. coli, but also eukaryotic cells such as cells of Saccharomyces cerevisiae, Pichia pastoris, phytopathogenic fungi, plants, frog oocytes and mammalian cell lines.

The present invention furthermore relates to polypeptides with the biological activity of ACATs which are encoded by the nucleic acids according to the invention.

The polypeptides according to the invention preferably comprise an amino acid sequence selected from

a) the sequence as shown in SEQ ID NO. 4,

b) sequences which have at least 60%, preferably 80% and especially preferably 90% identity with the sequences defined under a), and

c) sequences with the same biological activity as the sequences defined under a).

The term “polypeptides” as used in the present context refers not only to short amino acid chains which are generally referred to as peptides, oligopeptides or oligomers, but also to longer amino acid chains which are normally referred to as proteins. It encompasses amino acid chains which can be modified either by natural processes, such as post-translational processing, or by chemical prior-art methods. Such modifications may occur at various sites and repeatedly in a polypeptide, such as, for example, on the peptide backbone, on the amino acid side chain, on the amino and/or the carboxyl terminus. For example, they encompass acetylations, acylations, ADP ribosylations, amidations, covalent linkages to flavins, haem moieties, nucleotides or nucleotide derivatives, lipids or lipid derivatives or phosphatidylinositol, cyclizations, disulphide bridge formations, demethylations, cystine formations, formylations, gamma-carboxylations, glycosylations, hydroxylations, iodinations, methylations, myristoylations, oxidations, proteolytic processings, phosphorylations, selenoylations and tRNA-mediated amino acid additions.

The polypeptides according to the invention may exist in the form of “mature” proteins or as part of larger proteins, for example as fusion proteins. They can furthermore exhibit secretion or leader sequences, pro-sequences, sequences which allow simple purification, such as polyhistidine residues, or additional stabilizing amino acids. The proteins according to the invention may also exist in the form in which they are naturally present in their source organism, from which they can be obtained directly, for example.

The term “complete ACAT” as used in the present context describes an ACAT which is encoded by a complete coding region of a transcription unit starting with the ATG start codon and comprising all information-bearing exon regions of the gene encoding ACAT which is present in the source organism, and signals required for correct transcriptional termination.

In comparison with the corresponding regions of naturally occurring ACATs, the polypeptides according to the invention can have deletions or amino acid substitutions, as long as they still exert at least one biological activity of the complete ACATs. Conservative substitutions are preferred. Such conservative substitutions encompass variations, one amino acid being replaced by another amino acid from among the following group:

1. Small aliphatic residues, non-polar residues or residues of little polarity: Ala, Ser, Thr, Pro and Gly;

2. Polar, negatively charged residues and their amides: Asp, Asn, Glu and Gln;

3. Polar, positively charged residues: His, Arg and Lys;

4. Large aliphatic non-polar residues: Met, Leu, Ile, Val and Cys; and

5. Aromatic residues: Phe, Tyr and Trp.

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The present invention therefore also relates to the use of polypeptides which exert at least one biological activity of an ACAT and which comprise an amino acid sequence with at least 60%, preferably 80%, identity and especially preferably 95% identity with the Ustilago maydis sequence as shown in SEQ ID NO. 1 or SEQ ID NO. 2.

The term “biological activity of an ACAT” as used in the present context means the ability to catalyse the acetylation of acetyl-CoA.

The nucleic acids according to the invention can be prepared in the customary manner. For example, all of the nucleic acid molecules can be synthesized chemically. Short sections of the nucleic acids according to the invention can also be synthesized chemically, and such oligonucleotides can be radiolabelled or labelled with a fluorescent dye. The labelled oligonucleotides can also be used for screening cDNA libraries generated starting from mRNA from, for example, phytopathogenic fungi. Clones with which the labelled oligonucleotides hybridize are chosen for isolating the DNA fragments in question. After characterization of the DNA which has been isolated, the nucleic acids according to the invention are obtained in a simple manner.

Alternatively, the nucleic acids according to the invention can also be generated by means of PCR methods using chemically synthesised oligonucleotides.

The term “oligonucleotide(s)” as used in the present context refers to DNA molecules composed of 10 to 50 nucleotides, preferably 15 to 30 nucleotides. They are synthesised chemically and can be used as probes.

Moreover, host cells containing the nucleic acids according to the invention may be cultured under suitable conditions in order to prepare the polypeptides according to the invention, in particular the polypeptide encoded by the nucleic acid sequence as shown in SEQ ID NO. 2. The desired polypeptides can then be isolated in the customary manner from the cells or the culture medium. Alternatively, the polypeptides may be generated in in-vitro systems.

To prepare the Ustilago maydis ACAT according to the invention, it is possible, for example, to express the gene acc1 recombinantly in Escherichia coli and to prepare an enzyme preparation from E. coli cells.

One possible ACAT purification method is based on preparative electrophoresis, FPLC, HPLC (for example using gel filtration columns, reversed-phase columns or mildly hydrophobic columns), gel filtration, differential precipitation, ion-exchange chromatography or affinity chromatography.

A rapid method of isolating the polypeptides according to the invention which are synthesised by host cells using a nucleic acid to be used in accordance with the invention starts with expressing a fusion protein, where the fusion moiety may be purified in a simple manner by affinity purification. For example, the fusion moiety may be a six-His tag, in which case the fusion protein can be purified on a nickel-NTA affinity column. The fusion moiety can be removed by partial proteolytic cleavage, for example at linkers between the fusion moiety and the polypeptide according to the invention which is to be purified. The linker can be designed in such a way that it includes target amino acids, such as arginine and lycine residues, which define sites for trypsine cleavage. Standard cloning methods using oligonucleotides may be employed for generating such linkers.

Other purification methods which are possible are based, in turn, on preparative electrophoresis, FPLC, HPLC (e.g. using gel filtration columns, reversed-phase columns or mildly hydrophobic columns), gel filtration, differential precipitation, ion-exchange chromatography and affinity chromatography.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, especially preferably at least 100 times, higher than in a host cell preparation.

The polypeptides according to the invention may also be affinity-purified without fusion moieties with the aid of antibodies which bind to the polypeptides.

The present invention also relates to methods of finding chemical compounds which bind to ACAT and modify its properties. Owing to the important function of ACAT, modulators which affect the activity constitute novel fungicidal active compounds. Modulators may be agonists or antagonists, or inhibitors or activators.

The present invention likewise relates to the use of the polypeptides according to the invention in methods for finding chemical compounds which bind to ACAT and modify its properties.

Owing to the property of acting as inhibitors of fungal ACAT, in particular of ACAT of phytopathogenic fungi, methods which inhibit ACAT and which are found by means of suitable methods may also be used as optionally labelled competitors in methods of finding further inhibitors of fungal ACAT which need not belong to this group of compounds.

The use of the nucleic acids or polypeptides according to the invention in a method according to the invention makes it possible to find compounds which bind to the polypeptides according to the invention. The latter can then be used as fungicides, for example in plants, or as antimycotic active compounds in humans and animals. For example, host cells which contain the nucleic acids according to the invention and which express the corresponding polypeptides, or the gene products themselves, are brought into contact with a compound or a mixture of compounds under conditions which permit the interaction of at least one compound with the host cells, the receptors or the individual polypeptides.

In particular, the present invention relates to a method which is suitable for identifying fungicidal active compounds which bind to fungal polypeptides with the biological activity of an ACAT, preferably to ACAT from phytopathogenic fungi, especially preferably to ACAT from Ustilago, and polypeptides which are homologous thereto and which have the abovementioned consensus sequence. However, the methods can also be carried out with a polypeptide which is homologous to ACAT and which is derived from a species other than those mentioned herein. Methods which use other ACATs than the one according to the invention are part of the present invention.

A large number of assay systems for the purpose of assaying compounds and natural extracts are designed for high throughput numbers in order to maximize the number of substances assayed within a given period. Assay systems based on cell-free processes require purified or semi-purified protein. They are suitable for an “initial” assay, which aims mainly at detecting any possible effect of a substance on the target protein.

To find modulators, a synthetic reaction mix (for example in-vitro transcription products) or a cellular component such as a membrane, a compartment or any other preparation containing the polypeptides according to the invention are incubated together with a labelled substrate or ligand of the polypeptides in the presence and absence of a candidate molecule which can be an agonist or antagonist. The ability of the candidate molecule to increase or to inhibit the activity of the polypeptides according to the invention can be identified on the basis of increased or reduced binding of the labelled ligand or increased or reduced conversion of the labelled substrate. Molecules which bind well and which lead to an increased activity of the polypeptides according to the invention are agonists. Molecules which bind well and which inhibit the biological activity of the polypeptides according to the invention are good antagonists. They may also take the form of inhibitors of the abovementioned class of fungicidal substances, but entirely new classes of substances too may show this modulatory activity.

Detection of the biological activity of the polypeptides according to the invention can be improved by what is known as a reporter system. In this aspect, reporter systems comprise, but are not restricted to, colorimetric or fluorimetric substrates which are converted into a product, or a reporter gene which responds to changes in the activity or the expression of the polypeptides according to the invention, or other known binding assays. Conversely, it is possible to employ substrates which are converted into a colorimetrically or fluorimetrically detectable product. In this context, the report system may consist of several coupled reaction steps, which, finally, yield a product which can be detected in a suitable manner.

In a preferred embodiment, an assay system is used which, besides ACAT, exploits the activity of two further enzymes, namely malate dehydrogenase (MDH) and citrate synthase (CIT), so that the ACAT activity can be determined with reference to the increase in NADH concentration, which is determined photometrically. During the enzyme assay, the reactions shown in FIG. 3 take place. The reactions shown are reversible reactions, whose direction can be influenced by the substrates which are offered. The direction of the reaction which is required for the enzyme assay is indicted by the arrow. The course of MDH reaction is normally such that predominantly malate is formed from oxalacetate. However, coupling with the CIT reaction causes the consumption of the oxalacetate, the increased conversion of malate by MDH and thus the formation of NADH, which can be measured. The increase in NADH can be measured in a simple manner fluorimetrically (excitation at 340 nm/emission at 465 nm or excitation at 360 nm/emission at 460 nm).

Modulators which are found in this coupled assay system are checked in a second assay system, which only contains the enzymes MDH and CIT. Specific ACAT modulators are identified by the fact that they only lead to increased or reduced activity in the ACAT, MDH and CIT assay, but not in the assay which only contains MDH and CIT.

A further example of a method by which modulators of the polypeptides according to the invention can be found is a displacement assay in which the polypeptides according to the invention and a potential modulator are combined, under suitable conditions, with a molecule which is known to bind to the polypeptides according to the invention, such as a natural substrate or ligand or a substrate or ligand mimetic. The polypeptides according to the invention can themselves be labelled, for example fluorimetrically or colorimetrically, so that the number of the polypeptides which are bound to a ligand or which have undergone a conversion can be determined accurately. The efficacy of an agonist or antagonist can be determined in this manner.

Effects such as cell toxicity are, as a rule, ignored in these in-vitro systems. The assay systems check not only inhibitory, or suppressive, effects of the substances, but also stimulatory effects. The efficacy of a substance can be checked by concentration-dependent assay series. Control mixtures without test substances can be used for assessing the effects.

Owing to the host cells containing nucleic acids encoding ACAT and available with reference to the present invention, but also owing to the corresponding, homologous ACATs from other species which can be identified by reference to the present invention, the development of cell-based assay systems for identifying substances which modulate the activity of the polypeptides according to the invention, is made possible.

Another possibility of identifying substances which modulate the activity of the polypeptides according to the invention is what is known as the scintillation proximity assay (SPA), see EP 015 473. This assay system exploits the interaction of a polypeptide (for example U. maydis ACAT) with a radiolabelled ligand (for example a small organic molecule or a second radiolabelled protein molecule). Here, the polypeptide is bound to microspheres or beads which are provided with scintillating molecules. As the radioactivity declines, the scintillating substance in the microsphere is excited by the subatomic particles of the radiolabel, and a detectable photon is emitted. The assay conditions are optimized so that only those particles emitted from the ligand lead to a signal which are emitted by a ligand bound to the polypeptide according to the invention.

In one possible embodiment, the U. maydis ACAT is bound to the beads, either together with, or without, interacting or binding test substances. Test substances which can be used are, inter alia, fragments of the polypeptide according to the invention. For example, a radiolabelled ligand might be a labelled acetyl-CoA analogue which cannot be acetylated. When a binding ligand binds to the immobilized ACAT, this ligand should inhibit or nullify an existing interaction between the immobilized ACAT and the labelled ligand in order to bind itself in the zone of the contact area. Once binding to the immobilized ACAT has taken place, it can be detected with reference to a flash of light. Accordingly, an existing complex between an immobilized and a free, labelled ligand is destroyed by the binding of a test substance, which leads to a decline in the intensity of the light flash detected. In this case, the assay system takes the form of a complementary inhibition system.

Another example of such an assay system is what is known as the two-hybrid system. A specific example is what is known as the interaction trap. This system involves the genetic selection of interacting proteins in yeast (see, for example, Gyuris et al., 1993). The assay system is designed to detect the interaction of two proteins and to describe it by the generation of a detectable signal when interaction has taken place.

Such an assay system can also be adapted to testing large numbers of test substances within a given period.

The system is based on the construction of two vectors, the bait vector and the prey vector. A gene encoding an ACAT according to the invention or fragments thereof is cloned into the bait vector and then expressed as fusion protein with the LexA protein, a DNA-binding protein. A second gene encoding an interaction partner of ACAT is cloned into the prey vector, where is it expressed as fusion protein with the B42 prey protein. The two vectors are present in a Saccharomyces cerevisiae host which contains copies of LexA-binding DNA 5′ of a lacZ or HIS3 reporter gene. If interaction between the two fusion proteins takes place, transcription of the reporter gene is activated. If the presence of a test substance leads to inhibition of or interference with the interaction, the two fusion proteins are no longer capable of interacting, and the product of the reporter gene is no longer produced.

Another example of a method with which modulators of the polypeptides according to the invention can be found is a displacement test in which the polypeptides according to the invention and a potential modulator are brought together, under suitable conditions, with a molecule which is known to bind to the polypeptides according to the invention, such as a natural substrate or ligand, or a substrate mimetic or ligand mimetic.

The term “competitor” as used in the present context refers to the property of the compounds to compete with other, possibly yet to be identified, compounds for binding to ACAT to displace the latter, or being displaced by the latter, from the enzyme.

The term “agonist” as used in the present context refers to a molecule which accelerates or increases the activity of ACAT.

The term “antagonist” as used in the present context refers to a molecule which slows down or prevents the activity of ACAT.

The term “modulator” as used in the present context is the generic term for agonist or antagonist. Modulators can be small organochemical molecules, peptides or antibodies which bind to the polypeptides according to the invention. Moreover, modulators can be small organochemical molecules, peptides or antibodies which bind to a molecule which, in turn, binds to the polypeptides according to the invention, thus influencing their biological activity. Modulators can be natural substrates and ligands, or structural or functional mimetics of these.

The term “fungicide” or “fungicidal” as used in the present context is the generic term for substances for controlling phytopathogenic fungi and for substances for controlling fungi which are pathogenic for humans or animals. Thus, the term also extends to substances which can be used as antimycotics. In a preferred meaning, the term relates to substances for controlling phytopathogenic fungi.

The modulators are preferably small organochemical compounds.

Binding of the modulators to ACAT can modify the cellular processes in a manner which leads to the destruction of the phytopathogenic fungi treated therewith.

The present invention therefore also relates to modulators of ACAT from phytopathogenic fungi, which are found with the aid of a method described in the present application of identifying ACAT modulators.

The present invention furthermore comprises methods of finding chemical compounds which modify the expression of the polypeptides according to the invention. Such “expression modulators”, too, may constitute new fungicidal active compounds. Expression modulators can be small organochemical molecules, peptides or antibodies which bind to the regulatory regions of the nucleic acids encoding the polypeptides according to the invention. Moreover, expression modulators may be small organochemical molecules, peptides or antibodies which bind to a molecule which, in turn, binds to regulatory regions of the nucleic acids encoding the polypeptides according to the invention, thus influencing their expression. Expression modulators may also be antisense molecules.

The present invention likewise relates to the use of modulators of the polypeptides according to the invention or of expression modulators as fungicides.

The present invention likewise relates to expression modulators of ACATs which are found with the aid of the above-described method of finding expression modulators.

The methods according to the invention include high-throughput screening (HTS) and ultra-high-throughput screening (UHTS). Both host cells and cell-free preparations which comprise the nucleic acids and/or the polypeptides according to the invention may be used.

The invention furthermore relates to antibodies which bind specifically to the polypeptides according to the invention or fragments of these. Such antibodies are raised in the customary manner. For example, said antibodies may be produced by injecting a substantially immunocompetent host with a certain amount of a polypeptide according to the invention or a fragment thereof which is effective for antibody production, and subsequently obtaining this antibody. Furthermore, an immortalized cell line which produces monoclonal antibodies may be obtained in a manner known per se. The antibodies may be labelled with a detection reagent, if appropriate. Preferred examples of such a detection reagent are enzymes, radiolabelled elements, fluorescent chemicals or biotin. Instead of the complete antibody, fragments may also be employed which have the desired specific binding properties.

The nucleic acids according to the invention can likewise be used for generating transgenic organisms such as bacteria, plants or fungi, preferably for generating transgenic plants and fungi, especially preferably for generating transgenic fungi. These can be employed for example in assay systems which are based on an expression, of the polypeptides according to the invention or their variants, which deviates from the wild type. They furthermore include any transgenic plants or fungi in which the expression of the polypeptides according to the invention or variants of these is altered by modifying genes other than those described hereinabove or by modifying gene control sequences (for example promoter). The transgenic organisms are also of interest for (over)producing the polypeptide according to the invention; here, for example, fungi (for example yeast or Ustilago maydis) which show a higher degree of expression of the polypeptide according to the invention in comparison with their natural form are particularly suitable for use in methods (indeed also HTS methods) for identifying modulators of the polypeptide.

Transgenic organisms are understood as meaning organisms into whose genome heterologous (foreign) genes (transgenes) have been inserted stably with the aid of experimental techniques and expressed under suitable conditions.

The most developed vector system for generating transgenic plants is a plasmid from the bacterium Agrobacterium tumefaciens. In nature, A. tumefaciens infects plants and generates tumours termed crown galls. These tumours are caused by the Ti plasmid (tumour-inducing) of A. tumefaciens. The Ti plasmid incorporates part of its DNA, termed T-DNA, into the chromosomal DNA of the host plant. A means of removing the tumour-inducing regions from the DNA of the plasmid, but retaining its property of introducing genetic material into the plants, has been developed. Then, a foreign gene, for example one of the nucleic acids according to the invention, can be incorporated into the Ti plasmid with the aid of customary recombinant DNA techniques. The recombinant plasmid is then reinserted into A. tumefaciens, which can be then used for infecting a plant cell culture. However, the plasmid can also be inserted directly into the plants, where it incorporates itself into the chromosomes. Regeneration of such cells into intact organisms gives rise to plants containing the foreign gene and also expressing it, i.e. producing the desired gene product.

While A. tumefaciens infects dicotyledonous plants with ease, it is of limited use as vector for the transformation of monocotyledonous plants, which include a large number of agriculturally important crop plants such as maize, wheat or rice, since it does not infect these plants readily. Other techniques, for example “DNA guns”, what is known as the particle gun method, are available for the transformation of such plants. In this method, minute titanium or gold microspheres are fired into recipient cells or tissue, either by means of a gas discharge or by a powder explosion. The microspheres are coated with DNA of the genes of interest, whereby the latter reach the cells and are gradually detached and incorporated into the genome of the host cells.

Only a few of the cells which are exposed to the foreign hereditary material are capable of integrating it stably into their homologous hereditary material. In a tissue which is used for gene transfer, the non-transgenic cells predominate. During the regeneration into the intact plant, it is therefore necessary to apply a selection which provides an advantage for the transgenic cells. In practice, marker genes which are transferred into the plant cells are used for this purpose. The products of these genes inactivate an inhibitor, for example an antibiotic or herbicide, and thus allow the transgenic cells to grow on the nutrient medium supplemented with the inhibitor. However, genes which encode an enzyme which can then be detected are less problematic. These also include the polypeptide according to the invention, whose enzymatic activity can be detected as described in Example 2.

In the case of the transformation with A. tumefaciens, protoplasts (isolated cells without cell wall which, in culture, take up foreign DNA in the presence of certain chemicals or else when using electroporation) may be used instead of leaf segments. They are kept in tissue culture until a new cell wall has formed (for example approximately 2 days in the case of tobacco). Then, agrobacteria are added, and the tissue culture is continued. A simple method for the transient transformation of protoplasts with a DNA construct is the incubation in the presence of polyethylene glycol (PEG 4000).

DNA may also be introduced into cells by means of electroporation. This is a physical method for increasing the DNA uptake into live cells. Electrical pulses temporarily increase the permeability of a biomembrane without destroying the membrane.

DNA may also be introduced by microinjection. DNA is injected into the vicinity of the nucleus of a cell with the aid of glass capillaries. However, this is difficult in the case of plant cells, which have a rigid cell wall and a large vacuole.

A further possibility is to exploit ultrasound: when cells are sonicated with soundwaves above the frequency range of hearing in humans (above 20 kHz), a temporary permeability of the membranes is also observed. When carrying out this method, the amplitude of the soundwaves must be adjusted very precisely since, otherwise, the sonicated cells burst and are destroyed.

Transgenic fungi can be generated in the manner known per se to the skilled worker (see also Examples).

The invention thus also relates to transgenic plants or fungi which contain at least one of the nucleic acids according to the invention, preferably transgenic plants such as Arabidopsis species or transgenic fungi such as yeast species or Ustilago species, and their transgenic progeny. They also encompass the plant parts, protoplasts, plant tissues or plant propagation materials of the transgenic plants, or the individual cells, fungal tissue, fruiting bodies, mycelia and spores of the transgenic fungi which contain the nucleic acids according to the invention. Preferably, the transgenic plants or fungi contain the polypeptides according to the invention in a form which deviates from the wild type. However, those transgenic plants or fungi which are naturally characterized by only a very low degree of expression, or none at all, of the polypeptide according to the invention are also considered as being according to the invention.

Accordingly, the present invention likewise relates to transgenic plants and fungi in which modifications in the sequence encoding polypeptides with the activity of a ACAT have been generated and which have then been selected for the suitability for generating a polypeptide according to the invention and/or an increase or reduction, obtained by mutagenesis, in the biological activity or the amount of the polypeptide according to the invention which is present in the plants or fungi.

The term “mutagenesis” as used in the present context refers to a method of increasing the spontaneous mutation rate and thus of isolating mutants. In this context, mutants can be generated in vivo with the aid of mutagens, for example with chemical compounds or physical factors which are suitable for triggering mutations (for example base analogues, UV rays and the like). The desired mutants can be obtained by selecting towards a particular phenotype. The position of the mutations on the chromosomes can be determined in relation to other, known mutations by complementation and recombination analyses. Alternatively, mutations can also be introduced into chromosomal or extrachromosomal DNA in a directed fashion (in-vitro mutagenesis, site-directed mutagenesis, error-prone PCR and the like).

The term “mutant” as used in the present context refers to an organism which bears a modified (mutated) gene. A mutant is defined by comparison with the wild type which bears the unmodified gene.

The examples which follow now demonstrate that, surprisingly, ACAT is an essential enzyme in phytopathogenic fungi and furthermore that the enzyme is a suitable target protein for identifying fungicides, that it can be used in methods of identifying fungicidally active compounds, and that the ACAT modulators which have been identified in suitable methods can be used as fungicides.

Furthermore, obtaining this enzyme from Ustilago maydis and from Saccharomyces cerevisiae is described by way of example and, finally, the application of the present invention in the search for fungicidally active compounds is demonstrated.

The examples which follow are not limited to Ustilago maydis. Analogous methods and results are also obtained in connection with other phytopathogenic fungi.

### EXAMPLES

### Example 1

Functional Expression of the Saccharomyces cerevisiae ACAT Gene in E. coli

The coding sequence of the Saccharomyces cerevisiae ACAT (ERG10), which has been amplified via PCR, is changed into the expression vector pET-21b (Novagen) into the NdeI and XhoI cleavage sites so that a C-terminal His-tag is attached. Cells of the bacterial strain NovaBlue BL21(DE) (Novagen) are transformed with this His-tag ACAT construct (406 amino acids) and stored as long-term culture in glycerol at −80° C.

Isolation and Purification of ACAT from Saccharomyces cerevisiae

Cells from the long-term culture in glycerol are transferred to a fresh LB plate and streaked out to produce individual colonies. An overnight culture (approx. 2 ml) is established with one individual colony, and 20 μl of this culture are used to inoculate 250 ml of LB medium (1% tryptone, 0.5% yeast extract, 0.5% NaCl) supplemented with ampicillin (100 μg/ml) and incubated at 37° C. with shaking. After an OD₆₀₀=0.9 has been reached (after 4-6 h), expression of the polypeptide is induced by adding 1 mM IPTG (final concentration), and incubation of the culture is continued for 16 hours at 18° C. with shaking. The cells are harvested by centrifugation [8000 rpm≅10000 g, 4° C., JA 14 rotor, J2-21 centrifuge (Beckmann)] and frozen at −20° C.

The pellet (1.5 g) is taken up in 15 ml of break buffer (50 mM Tris/HCl, pH 8.0; 1 mM glutathione). 30 mg of lysozyme and 2.5 ml of 1% Triton X-100 in water are added and the mixture is incubated for 30 minutes at 28° C. The container is then transferred into a water/ethanol/ice bath, and the cells are disrupted by sonication (Branson Sonifier 250, use of the large sonifier head, output 50-55%, six times for 30 seconds each, with 1 minute cooling each time). After addition of 0.8 ml of 5 M sodium chloride solution (final concentration 0.2 M), cell debris is removed by spinning for 30 minutes (J2-21, JA20 rotor (Beckmann), 4° C., 10000 g). The supernatant contains the ACAT (crude extract).

The ACAT is isolated from the supernatant in portions of twice 10 ml by means of affinity chromatography (2 ml Qiagen Ni-NTA Superflow column, FPLC system from Pharmacia). Two buffers are used for this purpose:

Buffer A: 50 mM Tris/HCl pH 8.0, 1 mM glutathione

Buffer B: 50 mM Tris/HCl pH 8.0, 1 mM glutathione, 1 M imidazole

The chromatography is run using a gradient programme:

1) 10 column volumes buffer A

2) 10 column volumes buffer A:B 995:5 (final concentration 5 mM imidazole)

3) 10 column volumes buffer A:B 90:10 (final concentration 100 mM imidazole)

4) 5 column volumes buffer B (final concentration 1 M imidazole)

The protein elutes completely at a concentration of 100 mM imidazole. Under these conditions, the column is overloaded (protein also appears in the flow-through).

The protein (10 ml) is desalinated over four PD10 columns at 8° C. using 50 mM Tris/HCl pH 8.0, 1 mM glutathione, 10% glycerol.

This gives approximately 15 mg of ACAT with a purity of over 99% (Coomassie staining cf. FIG. 4).

Freezing of ACAT at −80° C. or −20° C. brings about an activity loss of approximately 15% or 20%, respectively. However, it may be stored on ice over several weeks without substantial loss of activity. The present example describes the stability of the enzyme and substrate solutions, which are stored separately at different temperatures.

### Example 2

Functional Expression of a EDNA Clone of the Ustilago maydis acc1 Gene in E. coli

The coding sequence of the U. maydis acc1 gene, which has been amplified via PCR, is cloned into the expression vector pET-21b (Novagen) into the NdeI and XhoI cleavage sites so that a C-terminal His-tag is attached. Cells of the bacterial strain NovaBlue BL21(DE) (Novagen) are transformed with this His-tag ACAT construct (408+2 amino acids owing to cloning of the C terminus+6 histidines=416 amino acids) and stored as long-term culture in glycerol at −80° C.

Isolation and Purification of ACAT from Ustilago maydis

Cells from the long-term culture in glycerol are transferred to a fresh LB plate and streaked out to produce individual colonies. An overnight culture (approx. 2 ml) is established with one individual colony, and 20 μl of this culture are used to inoculate 250 ml of LB medium (1% tryptone, 0.5% yeast extract, 0.5% NaCl) supplemented with ampicillin (100 μg/ml) and incubated at 37° C. with shaking. After an OD₆₀₀=0.9 has been reached (after 4-6 h), expression of the polypeptide is induced by adding 1 mM IPTG (final concentration), and incubation of the culture is continued for 16 hours at 18° C. with shaking. The cells are harvested by centrifugation [8000 rpm≅10000 g, 4° C., JA 14 rotor, J2-21 centrifuge (Beckmann)] and frozen at −20° C.

The pellet (1.5 g) is taken up in 15 ml of break buffer (50 mM Tris/HCl, pH 8.0; 1 mM glutathione). 30 mg of lysozyme and 2.5 ml of 1% Triton X-100 in water are added and the mixture is incubated for 30 minutes at 28° C. The container is then transferred into a water/ethanol/ice bath, and the cells are disrupted by sonication (Branson Sonifier 250, use of the large sonifier head, output 50-55%, six times for 30 seconds each, with 1 minute cooling each time). After addition of 0.8 ml of 5 M sodium chloride solution (final concentration 0.2 M), cell debris is removed by spinning for 30 minutes (J2-21, JA20 rotor (Beckmann), 4° C., 10000 g). The supernatant contains the ACAT (crude extract).

The Ustilago ACAT is isolated from the supernatant in portions by means of affinity chromatography and processed and used analogously to the yeast enzyme.

### Example 3

Enzyme Assay for Finding ACAT Modulators

5 μl portions of the substances to be assayed are introduced into a 384-well microtitre plate 5% (v/v) dimethyl sulphoxide. A solution of iodoacetamide, a known ACAT inhibitor, is used as control. The concentration of the substances is such that the final concentration of the substances in the assay carried out amounts to between 2 μM and 10 μM. 20 μl of enzyme solution (cooled to 1-2° C., composition: see hereinbelow) and 25 μl of substrate solution (cooled to 1-2° C., composition: see hereinbelow) are pipetted to the solution of the test substance. Depending on the specific activity of the enzymes involved, approximately 100-500 ng of total protein are employed. The reaction starts by addition of the substrate solution. Incubation is carried out at room temperature for 25 minutes. The decline in fluorescence is measured at λ_{Ex}=340 nm and λ_{Em}=465 nm or at λ_{Ex}=360 nm and λ_{Em}=460 nm.

The table which follows contains information on all of the substances used in the assay (stock solution and final concentrations):

| | | | Final | |
|---|---|---|---|---|
| | | | concentration | |
| | Stock solution | | in the assay | |
| Enzyme solution (20 μl): | | | | |
| 250 mM Tris/HCl pH 8.0 | 1 | M | 100 | mM |
| 25 mM magnesium chloride | 1 | M | 10 | mM |
| 2.5 mM (L)-malate | 100 | mM | 1 | mM |
| 6.25 mM dithiothreitol | 100 | mM | 2.5 | mM |
| 2.5 mM NAD⁺⁽¹⁾ | 25 | mM | 1 | mM |
| 3.375 μM coenzyme A | 0.45 | mM | 1.35 | μM |
| 2.5 mM glutathione | 100 | mM | 1 | mM |
| 12.5% glycerol | 50% | | 5% | |
| 250 μg/ml bovine serum albumine | [20 | mg/ml] | 100 | μg/ml |
| 1.56 ng/μl malate dehydrogenase | [10 | μg/ml] | 31.25 | ng/50 μl |
| 7.5 ng/μl citrate synthase | [10 | μg/ml] | 150 | ng/50 μl |
| 0.34 ng/μl ACAT | [0.917 | μg/ml] | 6.8 | ng/50 μl |
| Substrate solution (25 μl): | | | | |
| 160 μM acacCoA⁽²⁾ | 160 | μM | 80 | μM |
| Inhibitor solution (5 μl): | | | | |
| e.g. iodoacctamide | 100 | mM | 10 | mM |
| dimethyl sulphoxide | 5% | | 0.5% | |

⁽¹⁾NAD: nicotinamide-adenine-dinucleotide
⁽²⁾acacCoA: acetoacetyl-coenzyme A

20 μl of enzyme solution, 5 μl of inhibitor solution or solution of the test substance and 25 μl of substrate solution are used.

Substances which, in the above-described assay, have brought about an increase or reduction in the enzyme activity are tested in a second assay for modulation of MDH or CIT:

5 μl portions of the substances to be tested are introduced into a 384-well microtiter plate in 5% (v/v) dimethyl sulphoxide. The concentration of the substance is such that the final concentration of the substances in the assay carried out amounts to between 2 μM and 10 μM. 20 μl of enzyme solution (cooled to 1-2° C., composition: see hereinbelow) and 25 μl of substrate solution (cooled to 1-2° C., composition: see hereinbelow) are pipetted to the solute of the test substance. Depending on the specific activity of the enzymes involved, approximately 100-500 ng of total protein are employed. The reaction starts by addition of the substrate solution. Incubation is carried out at room temperature for 25 minutes. The decline in fluorescence is measured at λ_{Ex}=340 nm and λ_{Em}=465 nm or at λ_{Ex}=360 nm and λ_{Em}=460 nm.

The table which follows contains information on all of the substances used in the assay (stock solution and final concentrations):

| | | | Final | |
|---|---|---|---|---|
| | | | concentration | |
| | Stock solution | | in the assay | |
| Enzyme solution (20 μl): | | | | |
| 250 mM Tris/HCl pH 8.0 | 1 | M | 100 | mM |
| 25 mM magnesium chloride | 1 | M | 10 | mM |
| 2.5 mM (L)-malate | 100 | mM | 1 | mM |
| 6.25 mM dithiothreitol | 100 | mM | 2.5 | mM |
| 2.5 mM NAD⁺⁽¹⁾ | 25 | mM | 1 | mM |
| 3.375 μM coenzyme A | 0.45 | mM | 1.35 | μM |
| 2.5 mM glutathione | 100 | mM | 1 | mM |
| 12.5% glycerol | 50% | | 5% | |
| 250 μg/ml bovine serum albumine | [20 | mg/ml] | 100 | μg/ml |
| 1.56 ng/μl malate dehydrogenase | [10 | μg/ml] | 31.25 | ng/50 μl |
| 7.5 ng/μl citrate synthase | [10 | μg/ml] | 150 | ng/50 μl |
| Substrate solution (25 μl): | | | | |
| 320 μM acetyl-CoA | 320 | μM | 160 | μM |
| Solution of the test substance (5 μl): | | | | |
| test substance | | | 2 | μM to |
| | | | 10 | μM |
| dimethyl sulphoxide | 5% | | 0.5% | |

⁽¹⁾NAD: nicotinamide-adenine-dinucleotide

20 μl of enzyme solution, 5 μl of the solution of test substance and 25 μl of substrate solution are used.

### Example 4

The enzyme activity of ACAT as a function of time is shown in FIG. 5 (▪). The shape of the plot in the presence of an unspecific inhibitor is shown for comparison reasons (●).

### Example 5

Construction of the Hygromycin Knock-Out Cassette (hph Cassette)

Step 1

The bacterial hygromycin resistance gene (hph) is amplified from the plasmid pCM54 (cf. Tsukuda et al. 1988) by PCR with the primers hph-Nco/Bam (SEQ ID NO. 7) and hph-Stop (SEQ ID NO. 8) (PCR protocol of Innis et al. 1990; cycles: a) 1 cycle of 10 minutes at 94° C., b) 30 cycles of in each case 1 minute at 94° C., 1 minute at 60° C., 3 minutes at 72° C., c) 1 cycle of 10 minutes at 72° C.). This introduces different restriction cleavage sites at the two flanks of the hph gene: the Nco I cleavage site with the sequence 5′-C↓CATGG-3′ at the ATG start codon of the hph gene and the Not I cleavage site with the sequence 5′-GC↓GGCCGC-3′ one nucleotide behind the STOP codon of hph gene. The PCR product is subsequent restricted with the enzymes Nco I and Not I (New England Biolabs, conditions as specified by the manufacturer).

Step 2

In a subsequent step, this PCR product, which contains the hph gene, is linked with the agrobacterial NOS terminator. To this end, the plasmid potefSG (cf. Spellig et al. 1996), which contains firstly the sequence of the agrobacterial NOS terminator and secondly an exchangeable gene sequence for the sGFP gene, is restricted with the enzymes Nco I and Not I (New England Biolabs, conditions as specified by the manufacturer). The restriction liberates the fragment with the sGFP gene and exchanges it for the PCR fragment with the hph gene. Owing to this cloning step, the hph gene is flanked at the 3′ end by the agrobacterial NOS terminator to ensure effective termination of the transcription of hph.

Step 3

Starting from this plasmid, the hph gene together with the NOS terminator is amplified in a second PCR. To this end, the primers hph-Nco/Bam (SEQ ID NO. 7) and 3-hph (SEQ ID NO. 9) (PCR protocol of Innis et al. 1990; cycles: a) 1 cycle of 10 minutes at 94° C., b) 30 cycles of in each case 1 minute at 94° C., 1 minute at 60° C., 3 minutes at 72° C., c) 1 cycle of 10 minutes at 72° C.). Owing to this PCR, the cleavage sites for the restriction enzymes BamH I (with the sequence 5′-G↓GATCC-3′) and Nco I are generated at the 5′ end and the cleavage site Sfi I-b followed by a cleavage site of the enzyme Sac I (sequence 5′-GAGCT↓C-3′) is generated at the 3′ end. The PCR product is subsequently cloned directly into the plasmid pCR2.1 via TOPO cloning (using a kit from Invitrogen, conditions as specified by the manufacturer). This gives rise to the plasmid pBS-hph-Nos.

Step 4

Starting from the plasmid pCM54 (see above), a 550 bp fragment of the U. maydis hsp70 promoter is amplified, in a third PCR, by means of the primers 5-hsp (SEQ ID NO. 10) and hsp-Nco (SEQ ID NO. 11) (PCR protocol of Innis et al. 1990; cycles: a) 1 cycle of 10 minutes at 94° C., b) 30 cycles of in each case 1 minute at 94° C., 1 minute at 60° C., 3 minutes at 72° C., c) 1 cycle of 10 minutes at 72° C.). Owing this PCR, a cleavage site for the restriction enzyme Xho I (with the sequence 5′-C↓TCGAG-3′) followed directly by the cleavage site Sfi I-a is introduced at the 5′ end of the hsp70 promoter sequence. A cleavage site for the enzyme Nco I followed by a cleavage site for the enzyme HinDIII (with the sequence 5′-A↓AGCTT-3′) is generated at the 3′ end. Again, the PCR product is cloned directly into the plasmid pCR2.1 via TOPO cloning (using a kit from Invitrogen, conditions as specified by the manufacturer). This gives rise to the plasmid pBS-hsp. The correct sequences of the fragments generated via PCR are verified after cloning by means of sequencing (ABI 377 Automated Sequencer, universal and reverse primer, conditions as specified by the manufacturer using the standard universal and reverse primers).

Step 5

In a last step, i) the plasmid pBS-hph-Nos is cleaved with the enzymes Nco I and Sac I (New England Biolabs, conditions as specified by the manufacturer), thus giving rise to the fragment with the hph gene, the NOS terminator sequence and the cleavage site Sfi I-b, ii) the plasmid pBS-hsp is cleaved with the enzymes Xho I and Nco I (New England Biolabs, conditions as specified by the manufacturer), thus giving rise to the fragment with the hsp70 promoter sequence and the cleavage site Sfi I-a, iii) the commercially available plasmid pBSKSII (Stratagene) is cleaved with Xho I and Sac I (New England Biolabs, conditions as specified by the manufacturer), and iv) the three resulting fragments are ligated together (ligase from Roche, conditions as specified by the manufacturer), thus giving rise to the plasmid pBS-hhn. The hygromycin knock-out cassette can be obtained from this plasmid by restriction with Sfi I and employed in the method according to the invention.

Deletion of the Ustilago acc1 Gene, which Encodes an ACAT.

PCR is used to generate regions in each case approximately 750 bp in length which flank the ACAT gene (in the 3′ region) or extend into the second exon (in the 5′ region), using the primers with the sequences lacc11 (SEQ ID NO.12), lacc12 (SEQ ID NO. 13), racc11 (SEQ ID NO. 14) and racc12 (SEQ ID NO. 15).

The PCR is carried out under the following conditions: an initial denaturing step of 5 minutes at 94° C. is followed by 30 PCR cycles of in each case 1 minute at 94° C. (denaturing), 1 minute at 62° C. (hybridization) and 1 minute at 72° C. (polymerase reaction), followed by final incubation for 7 minutes at 72° C. (PCR protocol of Innis et al. 1990).

Owing to the PCR reaction, two different Sfi I cleavage sites, which are compatible with the Sfi I cleavage sites of the hph cassette, are introduced at the ends of the fragments which abut the acc1 gene or terminate therein. To this end, the primer lacc12 contains the sequence for the Sfi I-b cleavage site and 3 further nucleotides, in addition to the recognition sequence for the inner left-flanking region. The primer racc12 contains to this end the sequence for the Sfi I-a cleavage site and 3 further nucleotides, in addition to the recognition sequence for the inner right-flanking region.

The vector pBS-hhn and the PCR-generated fragments are restricted in a suitable manner with Sfi I, subsequently extracted with phenol, purified by gel electrophoresis and ligated with the hph cassette.

Following preciptation with ethanol, the PCR fragments are restricted with Sfi I (New England Biolabs, 20 units, 2 hours, conditions of the enzyme reaction as specified by the manufacturer).

After separation by electrophoresis in agarose gels, the fragments are purified with the gel elution kit Jetsorb from Genomed as specified by the manufacturer.

The flanks are ligated with the hph cassette in such a manner that 0.2 μg of each flank together with 0.2 μg of the 2 kb hph cassette are incubated with 2.5 units of ligase (Roche, conditions of the enzyme reaction as specified by the manufacturer).

Owing to the non-palindromic, different overhangs of the Sfi I cleavage sites, both the ligation of identical flanks and the ligation of different flanks are prevented. Ligation via the blunt ends of the fragments is ruled out since the primers employed in the PCR reaction are not phosphorylated. An overall product of 3.5 kb is amplified in a subsequent PCR with the corresponding external primers (lacc11 and racc12).

The PCR is carried under the following conditions: an initial denaturation step of 4 minutes at 94° C. is followed by 35 PCR cycles of in each case 1 minute at 94° C. (denaturation), 30 seconds at 60° C. (hybridization) and 4.5 minutes at 72° C. (polymerase reaction), with a final incubation for 7 minutes at 72° C.

The 3.5 kb PCR product is precipitated, taken up in water and employed directly for transforming the Ustilago maydis strain FBD11 (a1a2/b1b2) (description of the transformation see hereinbelow). 16 colonies are isolated from the transformation reaction and used for inoculating maize plants. Spores of the resulting tumours were subjected to segregation analysis. If the acc1 gene also has an essential function in Ustilago maydis, the following pattern in the segregation analysis is expected: haploide sporidia grow on medium without hygromycin B. No growth is observed in the presence of hygromycin B. In a typical result, the following pattern emerged: 11 sporidia grew in the absence of hygromycin B, but not in the presence of the antibiotic. One colony grew under both conditions. A copy of the acc1 gene and the hygromycin resistance gene were detected in this colony by PCR analysis. Only the acc1 gene was detected in the other strains. Since no candidates were found which lacked the acc1 gene and which are simultaneously resistant to hygromycin B, the acc1 gene has been classified as an essential gene of Ustilago maydis.

Generation of U. maydis Protoplasts, and Transformation

50 ml of U. maydis culture in YEPS medium are grown at 28° C. to a cell density of approx. 5×10⁷/ml (OD₆₀₀ 0.6 to 1.0). To this end, a stationary preculture is diluted 1:100, 1:300, 1:1000 in three steps and incubated in a baffle flask for approximately 16 hours at 28° C. and 200 rpm. After the desired cell density has been reached, the culture is spun down for 7 minutes at 2500 g. The cell pellet is resuspended in 25 ml of SCS buffer and again spun for 7 minutes 2500 g. The pellet is resuspended in 2 ml of SCS buffer supplemented with 2.5 mg/ml Novozym 234 (for example Novo-Biolabs). Protoplasts are released at room temperature; this is monitored under a microscope every 5 minutes. The protoplasts are subsequently mixed with 10 ml of SCS buffer and spun for 10 minutes at 1100 g. The supernatant is discarded. The pellet is carefully resuspended in 10 ml of SCS buffer, spun and subsequently washed with 10 ml of STC buffer, then resuspended in 500 μl of cold STC buffer and kept on ice.

15 μg of heparin and 50 μl of protoplasts (in ST buffer) are added in succession to not more than 7 μl of linear DNA (between 3 μg and 5 μg) and the mixture is cooled on ice for 10 minutes. Then, 500 μl of PEG4000 [40% (w/w) in STC buffer] are added, mixed carefully with the protoplast suspension and incubated for 15 minutes on ice.

To identify transformants, the transformation reaction is plated onto agar plates (YEPS medium supplemented with 1.5% agar, 1 M sorbitol and antibiotic; shortly before plating, this agar layer is covered with an equal volume of still fluid medium for agar plates without antibiotic). The result is determined after incubation at 28° C. for 3 to 4 days.

YEPS medium (cf. Tsukuda et al. 1988)

1% yeast extract, 2% Bacto peptone (Difco), 2% sucrose in water.

SCS buffer: 20 mM sodium citrate, 1.0 M sorbitol in water, pH 5.8.

STC buffer: 10 mM Tris/HCl (pH 7.5), 1.0 M sorbitol, 100 mM CaCl₂ in water.

Medium for agar plates for verifying the transformation

YEPS medium supplemented with 1.5% agar, 1.0 M sorbitol and antibiotic. Concentration of different antibiotics used in agar medium for verifying the transformation:

Phleomycin 80 μg/ml; Carboxin 4 μg/ml; Hygromycin 400 μg/ml; ClonNAT 300 μg/ml.

Information on the Sequence Listing

SEQ ID NO. 1: Genomic DNA sequence for the Ustilago maydis ACAT (acc1) (strain 521, DSM No. 14603).

SEQ ID NO. 2: DNA sequence of the counterstrand of the genomic DNA sequence of SEQ ID NO. 1, encoding the Ustilago maydis ACAT (strain 521, DSM No. 14603). The Ustilago maydis sequence contains two introns.

SEQ ID NO. 3: cDNA sequence encoding the Ustilago maydis ACAT.

SEQ ID NO. 4: Amino acid sequence encoded by the cDNA sequence of SEQ ID NO. 3 encoding Ustilago maydis ACAT.

SEQ ID NO. 5: DNA sequence encoding the Saccharomyces cerevisiae ACAT. The S. cerevisiae sequence contains no introns. The coding regions can be seen.

SEQ ID NO. 6: Amino acid sequence encoded by the exon of the sequence of SEQ ID NO. 5 encoding Saccharomyces cerevisiae ACAT.

SEQ ID NO. 7: DNA sequence of the primer hph-Nco/Bam.

SEQ ID NO. 8: DNA sequence of the primer hph-STOP.

SEQ ID NO. 9: DNA sequence of the primer 3-hph.

SEQ ID NO. 10: DNA sequence of the primer 5-hsp.

SEQ ID NO. 11: DNA sequence of the primer hsp-Nco.

SEQ ID NO. 12: DNA sequence of the primer lacc11

SEQ ID NO. 13: DNA sequence of the primer lacc12

SEQ ID NO. 14: DNA sequence of the primer racc11

SEQ ID NO. 15: DNA sequence of the primer racc12

### INFORMATION ON THE FIGURES

FIG. 1: Ergosterol biosynthesis part 1: from acetyl-CoA to squalene.

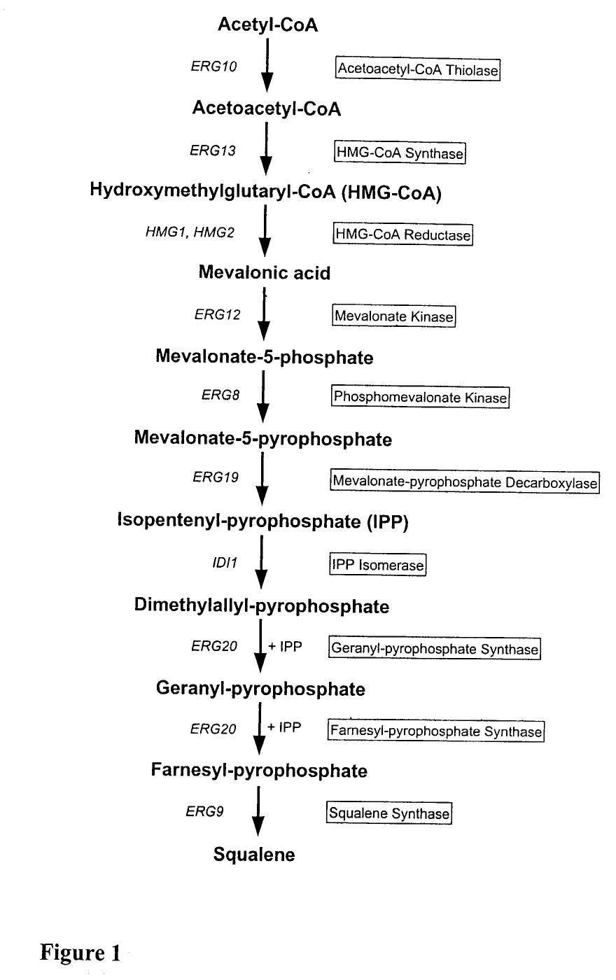

FIG. 2: Ergosterol biosynthesis part 2: from squalene to ergosterol.

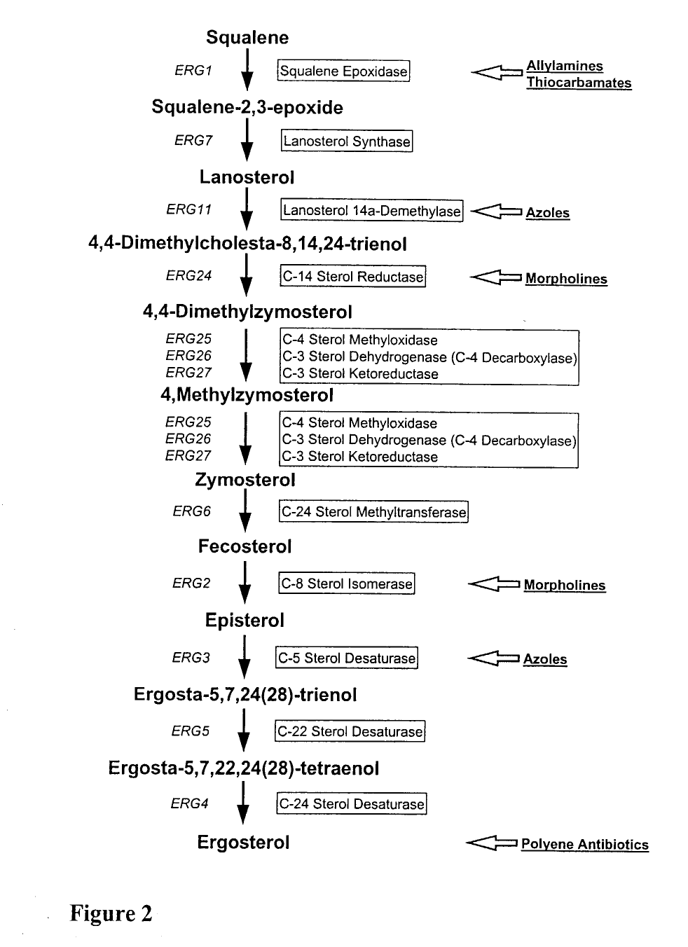

In FIG. 1 and FIG. 2, the individual intermediate steps starting from acetyl-CoA to ergosterol are shown in bold and black. The known enzymes are shown next to the reaction arrow in the box, the associated genes on the left of the reaction arrow in italics. Known targets (white arrow pointing to boxes) together with the relevant classes of active substances (bold and underlined) are also shown (cf. Daum et al., 1998 and Wills et al., 2000).

FIG. 3: Enzyme reactions in the ACAT enzyme assay

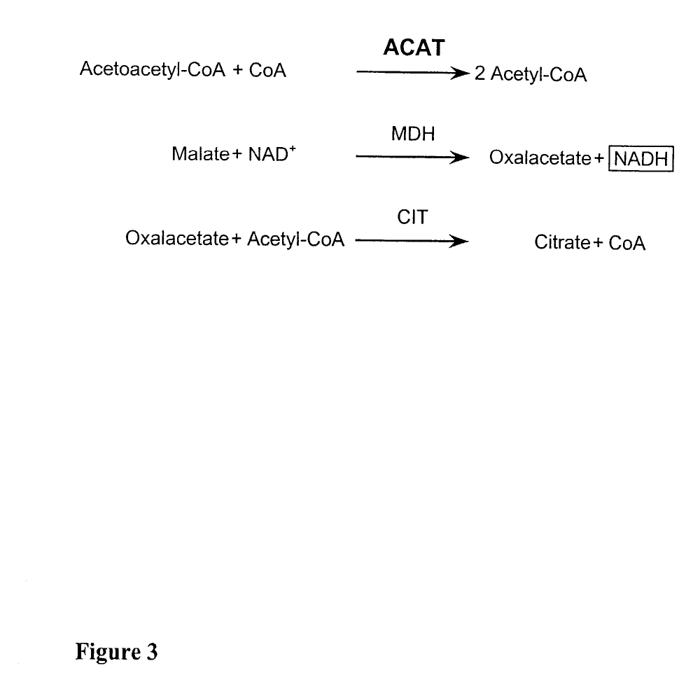

ACAT: Acetoacetyl-CoA thiolase

MDH: Malate dehydrogenase

CIT Citrate synthase

CoA Coenzyme A

NAD⁺ Nicotinamide adenine dinucleotide (oxidized form)

NADH Nicotinamide adenine dinucleotide (reduced form)

FIG. 4: SDS gel electrophoresis of individual fractions from the isolation of Saccharomyces cerevisiae ACAT

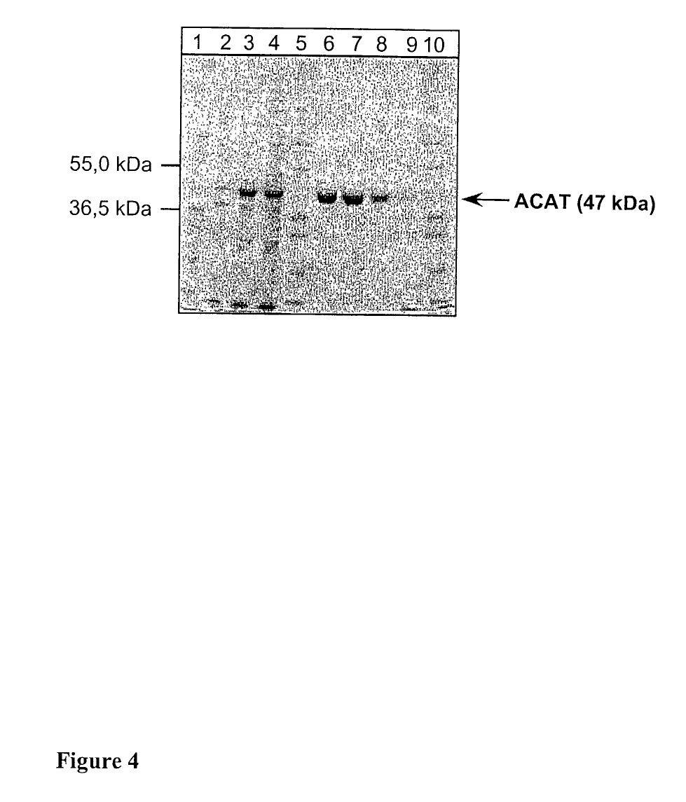

Lane 1: Size marker

Lane 2: Pellet (harvested cells), ≈2 μg

Lane 3: Crude extract (after cell disruption), 10 μg

Lane 4: Run-through, 10 μg

Lane 5: Size marker

Lane 6: ACAT fractions 6-8 after PD column, 5 μg

Lane 7: ACAT fractions 6-8 after FPLC, 5 μg

Lane 8: ACAT fraction 9 after FPLC, ≈1 μg

Lane 9: Eluate with buffer B (1 M imidazole)

Lane 10: Size marker

FIG. 5: Enzymatic activity of ACAT.

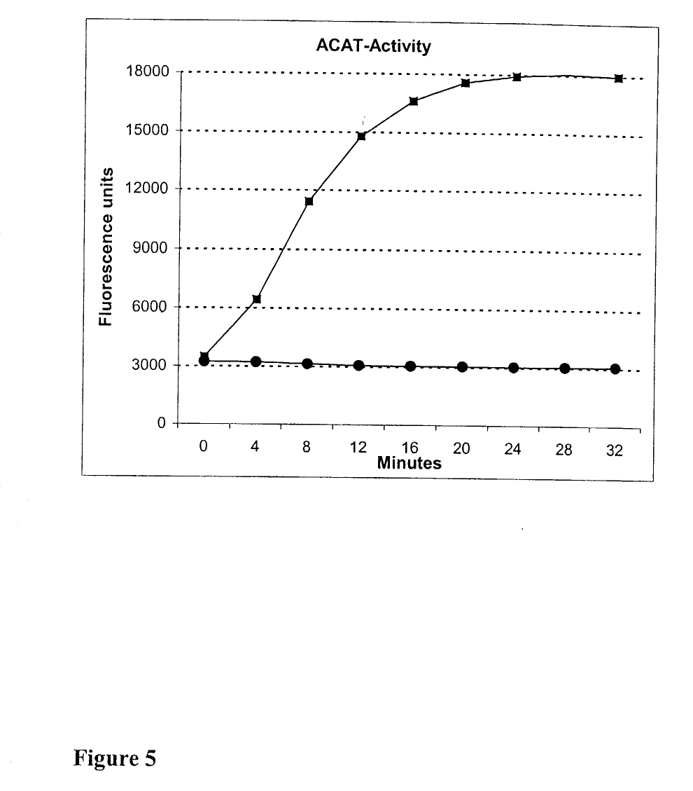

-▪- ACAT activity as a function of time

-●- ACAT activity in the presence of an inhibitor (10 mM iodoacetamide).

### REFERENCES

Altschul S F, Madden T L, Schaffer A A, Zhang J Z, Miller W, Lipman D J (1997): Gapped BLAST and PSI-BLAST generation of protein database search programs. Nucleic Acids Res. 25, 3389-3402.

Clinkenbeard K D, Sugiyama T, Lane M D (1975): Cytosolic acetoacetyl-CoA thiolase from chicken liver. Meth. Enzymol. 35, 167-73.

Daum G, Lees N D, Bard M, Dickson R (1998): Biochemistry, cell biology and molecular biology of lipids of Saccharomyces cerevisiae. Yeast 14, 1471-1510.

Dequin S, Gloeckler R, Herbert C J, Boutelet F (1988): Cloning, sequencing and analysis of the yeast S. uvarum ERG10 gene encoding acetoacetyl CoA thiolase. Curr. Genet. 13, 471-478.

Fukao T, Kamijo K, Osumi T, Fujiki Y, Yamaguchi S, Orii T, Hashimoto T (1989): Molecular cloning and nucleotide sequence of cDNA encoding the entire precursor of rat mitochondrial acetoacetyl-CoA thiolase. J. Biochem. (Tokyo) 106, 197-204.

Fukao T, Yamaguchi S, Kano M, Orii T, Fujiki Y, Osumi T, Hashimoto T (1990): Molecular cloning and sequence of the complementary DNA encoding human mitochondrial acetoacetyl-coenzyme A thiolase and study of the variant enzymes in cultured fibroblasts from patients with 3-ketothiolase deficiency. J. Clin. Invest. 86, 2086-2092.

Greenspan M D, Yudkovitz J B, Chen J S, Hanf D P, Chan M N, Chiang P Y C, Chabala J C, Alberts A W (1989): The Inhibition of Cytoplasmic Acetoacetyl-CoA Thiolase by a Triyne Carbonate (L-660,631). Biochem. Biophys. Res. Commun. 163, 548-553.

Gyuris J, Golemis E, Chertkov H, Brent R (1993): Cdi1, a human G1 and S phase protein phosphatase that associates with Cdk2. Cell 75, 791-803.

Hiser L, Basson M E, Rine J (1994): ERG10 from Saccharomyces cerevisiae Encodes Acetoacetyl-CoA Thiolase. J. Biol. Chem. 269, 31383-31389.

Innis M. A., Gelfand D. H., Sninsky J. J., White T. J., eds. (1990). PCR Protocols: A Guide to Methods and Applications. (Academic Press, San Diego, USA).

Kanayama N, Himeda Y, Atomi H, Ueda M, Tanaka A (1997): Expression of acetoacetyl-CoA thiolase isozyme genes of n-alkane-assimilating yeast, Candida tropicalis: isozymes in two intracellular compartments are derived from the same genes. J. Biochem. 122, 616-21.

Kornblatt J A, Rudney H (1971a): Two forms of acetoacetyl coenzyme A thiolase in yeast. I. Separation and properties. J. Biol. Chem. 246, 4417-4423.

Kornblatt J A, Rudney H (1971b): Two forms of acetoacetyl coenzyme A thiolase in yeast. II. Intracellular location and relationship to growth. J. Biol. Chem. 246, 4424-4430.

Kurahashi Y, Sakawa S, Kinbara T, Tanaka K, Kagabu S (1997): Biological Activity of Carpropamid (KTU 3616): A New Fungicide for Rice Blast Disease. J. Pestic. Sci. 22, 108-112.

Kurihara T, Ueda M, Tanaka A (1988): Occurrence and possible roles of acetoacetyl-CoA thiolase and 3-ketoacyl-CoA thiolase in peroxisomes of an n-alkane-grown yeast, Candida tropicalis. FEBS Lett. 229, 215-218.

Kurihara T, Ueda M, Kanayama N, Kondo J, Teranishi Y, Tanaka A (1992): Peroxisomal acetoacetyl-CoA thiolase of an n-alkane-utilizing yeast, Candida tropicalis. Eur. J. Biochem. 210, 999-1005.

Liebergesell M, Steinbuchel A (1993): Cloning and molecular analysis of the poly(3-hydroxybutyric acid) biosynthetic genes of Thiocystis violacea. Appl. Microbiol. Biotechnol. 38, 493-501.

Lottspeich F, Zorbas H (ed.) (1998): Bioanalytik. Spektrum Akademischer Verlag, Heidelberg, Berlin.

Modis Y, Wierenga R K (1999): A biosynthetic thiolase in complex with a reaction intermediate: the crystal structure provides new insights into the catalytic mechanism. Structure Fold Des. 7, 1279-90.

Peoples O P, Masamune S, Walsh C T, Sinskey A J (1987): Biosynthetic thiolase from Zoogloea ramigera.III. Isolation and characterization of the structural gene. J. Biol. Chem. 262, 97-102.

Spellig T., Bottin A., Kahmann R. (1996). Green fluorescent protein (GFP) as a new vital marker in the phytopathogenic fungus Ustilago maydis, Mol. Gen. Genet. 252, 503-509.

Tsukuda T, Carleton S, Fotheringham S, Holloman W K (1988). Isolation and characterization of an autonomously replicating sequence from Ustilago maydis, Mol. Cell. Biol. 8, 3703-3709.

Watanabe H, Yamaguchi S, Kimura M, Wakazono A, Song X Q, Fukao T, Orii T, Hashimoto T (1998): Practical Assay Method of Cytosolic Acetoacetyl-CoA Thiolase by Rapid Release of Cytosolic Enzymes from Cultured Lymphocytes Using Digitonin. Tohoku J. Exp. Med. 184, 29-38.

Wills E A, Redinbo M R, Perfect J R, Del Poeta M (2000): New potential targets for antifungal development. Emerging Therapeutic Targets 4, 1-32.

Yamagami S, Toshiya I, Yuji N, Akinori 0, Masamichi T (2001): Isolation and Characterization of Acetoacetyl-CoA Thiolase Gene Essential for n-Decane Assimilation in Yeast Yarrowia lipolytica. Biochem. Biophys. Res. Comm. 282, 832-838.

1. Nucleic acid encoding a polypeptide with the activity of an ACAT from phytopathogenic fungi.

2. Nucleic acid according to claim 1, characterized in that it encodes ACAT from basidiomycetes.

3. Nucleic acid according to claim 1 or 2, characterized in that it encodes ACAT from Ustilago.

4. Nucleic acids according to claim 1, 2 or 3, characterized in that they take the form of single-stranded or double-stranded DNA or RNA.

5. Nucleic acids according to one of claims 1 to 4, characterized in that they take the form of fragments of genomic DNA or the form of cDNA.

6. Nucleic acids according to one of claims 1 to 5, comprising a sequence selected from
a) a sequence as shown in SEQ ID NO. 1, SEQ ID NO. 2 and SEQ ID NO. 3,
b) sequences encoding a polypeptide comprising the amino acid sequence as shown in SEQ ID NO. 4
c) sequences which hybridize with the sequences defined under a) and/or b) at a hybridization temperature of 35-52° C.,
d) sequences with at least 60%, preferably 80%, especially preferably 90% and very especially preferably 95% identity with the sequences defined under a) and/or b),
e) sequences which are complementary to the sequences defined under a) and/or b), and
f) sequences which, owing to the degeneracy of the genetic code, encode the same amino acid sequence as the sequences defined under a) to e).

7. DNA construct comprising a nucleic acid according to one of claims 1 to 6 and a heterologous promoter.

8. Vector comprising a nucleic acid according to one of claims 1 to 6, or a DNA construct according to claim 7.

9. Vector according to claim 8, characterized in that the nucleic acid is linked operably to regulatory sequences which ensure the expression of the nucleic acid in prokaryotic or eukaryotic cells.

10. Host cell comprising a nucleic acid according to one of claims 1 to 6, a DNA construct according to claim 7 or a vector according to claim 8 or 9.

11. Host cell according to claim 10, characterized in that it takes the form of a prokaryotic cell.

12. Host cell according to claim 10, characterized in that it takes the form of a eukaryotic cell.

13. Polypeptide with the biological activity of an ACAT, which polypeptide is encoded by a nucleic acid according to one of claims 1 to 6.

14. Polypeptide with the biological activity of an ACAT selected from
a) the sequence as shown in SEQ ID NO. 4,
b) sequences which have at least 60%, preferably 80% and especially preferably 90% identity with the sequences defined under a), and
c) sequences which have the same biological activity as the sequences defined under a).

15. Antibody which binds specifically to a polypeptide according to claim 13 or 14.

16. Method of generating a nucleic acid according to one of claims 1 to 6, comprising the following steps:
(a) full chemical synthesis in a manner known per se, or
(b) chemical synthesis of oligonucleotides, labelling the oligonucleotides, hybridizing the oligonucleotides with DNA of a genomic library or a cDNA library generated from genomic DNA or mRNA from fungal cells, selecting positive clones, and isolating the hybridizing DNA from positive clones, or
(c) chemical synthesis of oligonucleotides and amplification of the target DNA by means of PCR.

17. Method of generating a polypeptide according to claim 13 or 14, which comprises
(a) culturing a host cell according to one of claims 10 to 12 under conditions which ensure the expression of the nucleic acid according to one of claims 1 to 6, or
(b) expressing a nucleic acid according to one of claims 1 to 6 in an in-vitro system, and
(c) obtaining the polypeptide from the cell, the culture medium or the in-vitro system.

18. Method of finding a chemical compound which binds to a polypeptide according to claim 13 or 14 and/or which modulates the activity of this polypeptide, which comprises the following steps:
(a) bringing a host cell according to one of claims 10 to 12 or a polypeptide according to claim 13 or 14 into contact with a chemical compound or a mixture of chemical compounds under conditions which permit the interaction of a chemical compound with the polypeptide, and
(b) identifying the chemical compound which binds specifically to the polypeptide and/or which modulates the activity of this polypetide.

19. Method of finding a compound which modifies the expression of polypeptides according to claim 13 or 14, which comprises the following steps:
(a) bringing a host cell according to one of claims 10 to 12 into contact with a chemical compound or a mixture of chemical compounds,
(b) determining the polypeptide concentration, and
(c) identifying the compound which specifically influences the expression of the polypeptide

20. Use of fungal ACAT, nucleic acids encoding it, or DNA constructs or host cells containing these nucleic acids for finding fungicidal active compounds.

21. Use of a modulator of a polypeptide with the biological activity of an ACAT as fungicide.

22. Use of a modulator of a polypeptide with the biological activity of an ACAT for preparing a composition for controlling harmful fungi.

23. Modulators which are identified by a method according to claim 18 or 19.

24. Fungicidally active substances which are found by means of a method according to claim 18 or 19.

25. Use of a nucleic acid according to one of claims 1 to 6, of a DNA construct according to claim 7 or of a vector according to claims 8 or 9 for generating transgenic plants and fungi.

26. Transgenic plants, plant parts, protoplasts, plant tissue or plant propagation materials, characterized in that, after introduction of a nucleic acid according to one of claims 1 to 6, of a DNA construct according to claim 7 or of a vector according to claims 8 or 9, the intracellular concentration of a polypeptide according to claim 13 or 14 is increased or reduced in comparison with the corresponding wild-type cells.

27. Transgenic fungi, fungal cells, fungal tissue, fruiting bodies, mycelia and spores, characterized in that, after introduction of a nucleic acid according to one of claims 1 to 6, of a DNA construct according to claim 7 or of a vector according to claims 8 or 9, the intracellular concentration of a polypeptide according to claim 13 or 14 is increased or reduced in comparison with the corresponding wild-type cells.

28. Plants, plant parts, protoplasts, plant tissue or plant propagation materials, characterized in that they contain a polypeptide according to claims 13 or 14 whose biological activity or expression pattern is modified in comparison with the corresponding endogenous polypeptides.

29. Method of generating plants, plant parts, protoplasts, plant tissues or plant propagation materials according to claim 28, characterized in that a nucleic acid according to one of claims 1 to 6 is modified by endogenous mutagenesis.

30. Fungi, fungal cells, fungal tissue, fruiting bodies, mycelia and spores, characterized in that they contain a polypeptide according to claim 13 or 14 whose biological activity or expression pattern is modified in comparison with the corresponding endogenous polypeptides.

31. Method of generating fungi, fungal cells, fungal tissue, mycelia and spores according to claim 30, characterized in that a nucleic acid according to one of claims 1 to 6 is modified by endogenous mutagenesis.


SEQUENCE LISTING (15 sequences)

SEQ ID NO: 1 - DNA, 1911 bp, Ustilago maydis
gttctcacgcttgataatgatagcagaagcgccaccaccaccgttgcagacaccagcgcagccgtattggccgggcttgagcgcgtgggccagcgtaacaacaatcctagcacctgaagaaccgatagggtgaccgagtgaaacaccaccaccgagaacgttgaccttgctagcgtcgaggccgagcatctggttgttagccaacgcgacagccgagaaagcctcgttgatttcgaacagagcaatgtcgtcctttgtcaaaccggctcgctcgagcgccttgggaatggcataagcgggcgcaatggggaagtcgatcggtgcacatgctgcatcggcaaacgctacaatcttggccaaaggcttaatgcccaacttggccacctcggcctcggaagctagcacaacggccgaagcaccgtcgttgagcgtcgacgcattggcagcagtgatggttccgttcttgtcgaacaccggtcgaagcgatgggatcttttcgagcttgacgttcttgtactcctcgtcttcgctgatcacgacatcgcccttcttgtccgagatggtgacaggagcgatctcgttagcgaatgcattggccttccatgcctcggcggagcgacggtacgattcaatcgcaaaggcgtcctgctgctcacgcgtaattgagagcttcttggcagtgttctcggcacagttgcccatggccacctggttgtagacatcgtgcagaccgtccttgacgatcgcatcggtcgcctggacatggccgtaggtgttgcctcggggaagatagtagctgttcgaaacaaacgaaacaaaagaaacaaaaagggaaaaatgtcagacaagagcttcctgagacagactcaactgccgccgttggggtgttcactcccctgtagctctctttcgaaaggcaatgtcgcgacgccaacagcctgtgcgaatgcaactatgggcctcctcatatgtgctagttttcgcttgtgatccttgatcccaagctgtagcctggcgtcgactggcgccgcgcatgacacccgagtcgagagtctcgggccaggcgggcaaaccaacgtccgaacgacattttccccacatgtactggaatgttaggccgacaccacgaattcgatccgaacggcaaataaaaatgcgggtgtaccggtccagcttttgcccttgccaagagttttgcttgggttgagccgtctaggccagagcaccaaacaaacgcttcgaagtagcaacctcagcgcacgctccccctcttctgcagtagagacatgctttctctaaagctgatgcaatgagagcctggataacgatacttttgtcggtacttacggggcgttagacatagactccatgccaccggcgaccatgatactgcgctggccgagagcaatgttctgggcggcgagcgagatggccttcataccggaagcgcacaccttgttaatggtggtagcctcggtggtgtcggggcagccagccttgagcgcaacctgacgggcgggagcctgaccgacgttgccttggagcacattgcccatgtagacctcctcgatctggtcgggcttgagaccggcacgttggatggctgcctttacggccaccacaccgagctcaggagcggtggcctttttgagcacaccgttgaacgagccgatgggcgttcgggcggcggaaacgatgaagacgtcgttgatttgtgactaatgtgtccatcgttgatgccgtttgtagagcgtagaggagaggggtggtatcgagaagtcagtcaatctggatgttgcgggcaccggagtacgtgtgtgtaggtgcaactttgcgactgcaatgcagacttaccataattctagctgtctgagaaagtcgtcggacggcggtttggtagaaagctggcat

SEQ ID NO: 2 - DNA, 1914 bp, Ustilago maydis
atgccagctttctaccaaaccgccgtccgacgactttctcagacagctagaattatggtaagtctgcattgcagtcgcaaagttgcacctacacacacgtactccggtgcccgcaacatccagattgactgacttctcgataccacccctctcctctacgctctacaaacggcatcaacgatggacacattagtcacaaatcaacgacgtcttcatcgtttccgccgcccgaacgcccatcggctcgttcaacggtgtgctcaaaaaggccaccgctcctgagctcggtgtggtggccgtaaaggcagccatccaacgtgccggtctcaagcccgaccagatcgaggaggtctacatgggcaatgtgctccaaggcaacgtcggtcaggctcccgcccgtcaggttgcgctcaaggctggctgccccgacaccaccgaggctaccaccattaacaaggtgtgcgcttccggtatgaaggccatctcgctcgccgcccagaacattgctctcggccagcgcagtatcatggtcgccggtggcatggagtctatgtctaacgccccgtaagtaccgacaaaagtatcgttatccaggctctcattgcatcagctttagagaaagcatgtctctactgcagaagagggggagcgtgcgctgaggttgctacttcgaagcgtttgtttggtgctctggcctagacggctcaacccaagcaaaactcttggcaagggcaaaagctggaccggtacacccgcatttttatttgccgttcggatcgaattcgtggtgtcggcctaacattccagtacatgtggggaaaatgtcgttcggacgttggtttgcccgcctggcccgagactctcgactcgggtgtcatgcgcggcgccagtcgacgccaggctacagcttgggatcaaggatcacaagcgaaaactagcacatatgaggaggcccatagttgcattcgcacaggctgttggcgtcgcgacattgcctttcgaaagagagctacaggggagtgaacaccccaacggcggcagttgagtctgtctcaggaagctcttgtctgacatttttccctttttgtttcttttgtttcgtttgtttcgaacagctactatcttccccgaggcaacacctacggccatgtccaggcgaccgatgcgatcgtcaaggacggtctgcacgatgtctacaaccaggtggccatgggcaactgtgccgagaacactgccaagaagctctcaattacgcgtgagcagcaggacgcctttgcgattgaatcgtaccgtcgctccgccgaggcatggaaggccaatgcattcgctaacgagatcgctcctgtcaccatctcggacaagaagggcgatgtcgtgatcagcgaagacgaggagtacaagaacgtcaagctcgaaaagatcccatcgcttcgaccggtgttcgacaagaacggaaccatcactgctgccaatgcgtcgacgctcaacgacggtgcttcggccgttgtgctagcttccgaggccgaggtggccaagttgggcattaagcctttggccaagattgtagcgtttgccgatgcagcatgtgcaccgatcgacttccccattgcgcccgcttatgccattcccaaggcgctcgagcgagccggtttgacaaaggacgacattgctctgttcgaaatcaacgaggctttctcggctgtcgcgttggctaacaaccagatgctcggcctcgacgctagcaaggtcaacgttctcggtggtggtgtttcactcggtcaccctatcggttcttcaggtgctaggattgttgttacgctggcccacgcgctcaagcccggccaatacggctgcgctggtgtctgcaacggtggtggtggcgcttctgctatcattatcaagcgtgagaactaa

SEQ ID NO: 3 - DNA, 1227 bp, Ustilago maydis
  CDS (1)..(1224)
atgccagctttctaccaaaccgccgtccgacgactttctcagacagctagaattatgtcacaaatcaacgacgtcttcatcgtttccgccgcccgaacgcccatcggctcgttcaacggtgtgctcaaaaaggccaccgctcctgagctcggtgtggtggccgtaaaggcagccatccaacgtgccggtctcaagcccgaccagatcgaggaggtctacatgggcaatgtgctccaaggcaacgtcggtcaggctcccgcccgtcaggttgcgctcaaggctggctgccccgacaccaccgaggctaccaccattaacaaggtgtgcgcttccggtatgaaggccatctcgctcgccgcccagaacattgctctcggccagcgcagtatcatggtcgccggtggcatggagtctatgtctaacgccccctactatcttccccgaggcaacacctacggccatgtccaggcgaccgatgcgatcgtcaaggacggtctgcacgatgtctacaaccaggtggccatgggcaactgtgccgagaacactgccaagaagctctcaattacgcgtgagcagcaggacgcctttgcgattgaatcgtaccgtcgctccgccgaggcatggaaggccaatgcattcgctaacgagatcgctcctgtcaccatctcggacaagaagggcgatgtcgtgatcagcgaagacgaggagtacaagaacgtcaagctcgaaaagatcccatcgcttcgaccggtgttcgacaagaacggaaccatcactgctgccaatgcgtcgacgctcaacgacggtgcttcggccgttgtgctagcttccgaggccgaggtggccaagttgggcattaagcctttggccaagattgtagcgtttgccgatgcagcatgtgcaccgatcgacttccccattgcgcccgcttatgccattcccaaggcgctcgagcgagccggtttgacaaaggacgacattgctctgttcgaaatcaacgaggctttctcggctgtcgcgttggctaacaaccagatgctcggcctcgacgctagcaaggtcaacgttctcggtggtggtgtttcactcggtcaccctatcggttcttcaggtgctaggattgttgttacgctggcccacgcgctcaagcccggccaatacggctgcgctggtgtctgcaacggtggtggtggcgcttctgctatcattatcaagcgtgagaactaa

SEQ ID NO: 4 - protein, 408 aa, Ustilago maydis
MPAFYQTAVRRLSQTARIMSQINDVFIVSAARTPIGSFNGVLKKATAPELGVVAVKAAIQRAGLKPDQIEEVYMGNVLQGNVGQAPARQVALKAGCPDTTEATTINKVCASGMKAISLAAQNIALGQRSIMVAGGMESMSNAPYYLPRGNTYGHVQATDAIVKDGLHDVYNQVAMGNCAENTAKKLSITREQQDAFAIESYRRSAEAWKANAFANEIAPVTISDKKGDVVISEDEEYKNVKLEKIPSLRPVFDKNGTITAANASTLNDGASAVVLASEAEVAKLGIKPLAKIVAFADAACAPIDFPIAPAYAIPKALERAGLTKDDIALFEINEAFSAVALANNQMLGLDASKVNVLGGGVSLGHPIGSSGARIVVTLAHALKPGQYGCAGVCNGGGGASAIIIKREN

SEQ ID NO: 5 - DNA, 1197 bp, Saccharomyces cerevisiae
  CDS (1)..(1194)
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 6 - protein, 398 aa, Saccharomyces cerevisiae
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 7 - DNA, 25 bp, Primer hph-Nco/Bam
gcggatcccatggaaaagcctgaac

SEQ ID NO: 8 - DNA, 32 bp, Primer hph-stop
gggcggccgctctattcctttgccctcggacg

SEQ ID NO: 9 - DNA, 44 bp, Primer 3-hph
cggagctcggccactcaggcctcatgtttgacagcttatcatcg

SEQ ID NO: 10 - DNA, 45 bp, Primer 5-hsp
cgctcgaggcctagatggccgaacgtggtaactaccagcgagttc

SEQ ID NO: 11 - DNA, 35 bp, Primer hsp-Nco
cggaagcttccatggccccccgggatctggcaacg

SEQ ID NO: 12 - DNA, 23 bp, Primer lacc11
gaagattcgtgattgaggaatcc

SEQ ID NO: 13 - DNA, 43 bp, Primer lacc12
gtcggcctgagtggccgttgatttgtgactaatgtgtccatcg

SEQ ID NO: 14 - DNA, 41 bp, Primer racc11
gacggccatctaggccactaataagctgctcgcttcatgac

SEQ ID NO: 15 - DNA, 23 bp, Primer racc12
gaccgtcatggatcgaaccatgg